(19) United States
(12) Patent Application Publication
(10) Pub. No.: US 2022/0010301 A1
(43) Pub. Date: Jan. 13, 2022

(54) METHODS OF SELECTING FUNCTIONAL INTERFACE MIMICS, AND COMPOSITIONS THEREOF

(71) Applicant: Cowper Sciences Inc., Chandler, AZ (US)

(72) Inventors: Mohan SRINIVASAN, Chandler, AZ (US); Matthew GREVING, Chandler, AZ (US)

(21) Appl. No.: 17/294,345

(22) PCT Filed: Nov. 13, 2019
(86) PCT No.: PCT/US2019/061196

§ 371 Date: May 14, 2021

Related U.S. Application Data
(60) Provisional application No. 62/767,426, filed on Nov. 14, 2018.

(51) Int. Cl. C12N 15/10; C07K 7/06; C07K 7/08

(52) U.S. Cl. CPC C12N 15/1055; C07K 7/06; A61K 39/00; C07K 2319/00; C07K 7/08

(57) ABSTRACT

Provided herein are functional interface mimics (FIMs), which mimic a functional interface of an interface protein. The FIMs comprise at least one peptide and at least one linking moiety, such as a linker or a cross-link. In some embodiments, the FIMs comprise at least two peptides connected by at least one linker. In some embodiments, the FIM is an immunogen. Also provided herein are methods of selected an FIM, and methods of producing an antibody using an FIM.

### CROSS-REFERENCE

This application claims the benefit of U.S. Provisional Application No. 62/767,426, filed Nov. 14, 2018, the entire disclosure of which is incorporated herein by reference.

### BACKGROUND

Mimotopes, which are peptides that functionally mimic a binding site but which may have structural differences from the target of interest, have been developed as molecules that may share characteristics with a surface of a protein of interest. Mimotopes of a target protein can be used, for example, to raise antibodies that recognize the target protein. Mimotope discovery is a largely stochastic process that is done by screening highly diverse random libraries. One challenge is that while selected mimotopes may retain certain desirable functionalities, they are typically so divergent from the native protein-protein interface that the mimotope can have unpredictable, undesirable characteristics. For example, using mimotopes as immunogens can result in selected antibodies having promiscuity as a result of the divergent mimotope structure.

### BRIEF SUMMARY

The present disclosure relates generally to peptide molecules, including polypeptide molecules, and more specifically to peptide molecules that mimic a target molecule interface, for example, surface or other portion of a nucleic acid, ribozyme, and/or protein, that interfaces or interacts with a binding partner or capture molecule. For example, the peptide molecules disclosed herein include proteins that interface with another molecule (referred to herein as interface proteins), including another protein, such as a cognate binding partner, a receptor, or an enzyme, for example. In some instances, the peptide molecules disclosed herein that mimic the functional interfaces of target molecules comprise peptide molecules comprising a linking moiety. In yet other instances, the peptide or polypeptide molecules disclosed herein may be combinatorial peptide molecules. In yet other instances, the peptide molecules disclosed herein may be fused or combined with other proteins or carrier molecules.

In some aspects, provided herein is a functional interface mimic (FIM), comprising at least one peptide that exhibits at least one characteristic of a target molecule, including an interface protein, and at least one linking moiety; and wherein the functional interface mimic exhibits at least one characteristic of the interface protein. In some embodiments, each linking moiety is independently a linker or a cross-link.

In some embodiments, the FIM comprises at least two peptides connected by at least one linker, wherein each peptide independently exhibits at least one characteristic of a target molecule, including an interface protein; and wherein the functional interface mimic exhibits at least one characteristic of the interface protein.

In other aspects, provided herein is a method of generating an antibody specific for a target, comprising injecting into an animal an FIM, wherein the FIM is an immunogen.

In still other aspects, provided herein is a population of FIM candidates, wherein a plurality of the candidates each independently comprise at least two peptides connected by at least one linker; wherein each peptide independently exhibits at least one characteristic of a target molecule, including an interface protein; and wherein the functional interface mimic (FIM) exhibits at least one characteristic of the interface protein.

In further aspects, provided herein is a method of selecting an FIM, the method comprising contacting a peptide library with a protein or fragment thereof, wherein the peptide library comprises a plurality of peptides, and wherein the protein or fragment thereof binds with at least two peptides; selecting at least a portion of peptides based on binding with the protein or fragment thereof that contacted the peptide library; generating a combinatorial library comprising a plurality of FIM candidates, wherein each candidate independently comprises at least two selected peptides connected by at least one linker; contacting the combinatorial library with a protein or fragment thereof, wherein the protein or fragment thereof binds with at least one candidate; and selecting an FIM from the combinatorial library based on binding with the protein or fragment thereof that contacted the combinatorial library.

In still other aspects, provided herein is an FIM produced by contacting a peptide library with a protein or fragment thereof, wherein the peptide library comprises a plurality of peptides, and wherein the protein or fragment thereof binds with at least two peptides; selecting at least a portion of peptides based on binding with the protein or fragment thereof that contacted the peptide library; generating a combinatorial library comprising a plurality of FIM candidates, wherein each candidate independently comprises at least two selected peptides connected by at least one linker; contacting the combinatorial library with a protein or fragment thereof, wherein the protein or fragment thereof binds with at least one candidate; and selecting the FIM from the combinatorial library based on binding with the protein or a fragment thereof that contacted the combinatorial library.

In some embodiments of the FIMs, FIM populations, or methods of selecting or making FIMs provided herein, the FIM is an immunogen, an antagonist, an agonist, or a reagent. In some embodiments, the FIM is an immunogen. In certain embodiments, the FIM shares at least one characteristic with a target molecule, including an interface protein. For example, the interface protein has a cognate binding partner, and the FIM exhibits binding with the cognate binding partner that is within at least one order of magnitude of the interface protein-cognate binding partner binding. In some embodiments, the FIM comprises two to twelve peptides. In some embodiments of the methods, population, or FIM provided herein, each peptide independently comprises two to forty amino acids. In some embodiments, the protein or fragment thereof that contacts the peptide library is the same protein or fragment thereof that contacts the combinatorial library.

### DESCRIPTION OF THE FIGURES

The present application can be understood by reference to the following description taken in conjunction with the accompanying figures.

FIG. 1 is a diagram illustrating the development of one exemplary embodiment of an FIM, wherein the FIM shares sequence similarity with a contiguous portion of a functional interface.

FIG. 2 is a diagram illustrating the development of one exemplary embodiment of an FIM, wherein the FIM shares sequence similarity with discontiguous portions of a functional interface.

FIG. 3 is an exemplary graph of a molecular dynamics simulation of FIM structural flexibility relative to a target interface crystal structure.

FIG. 4 is the simulated structure of an exemplary FIM comprising at least two peptides connected by at least one linker. The peptides of the FIM have amino acid residue similarities to the target functional interface (cognate interface motifs 1 and 2), and also comprise amino acid residues that stabilize the FIM.

### DETAILED DESCRIPTION

The present disclosure relates generally to peptide molecules, including polypeptide molecules, and more specifically to peptide molecules that mimic a target molecule interface, for example, surface or other portion of a nucleic acid, ribozyme, and/or protein, that interfaces or interacts with a binding partner or capture molecule. For example, the peptide molecules disclosed herein include proteins that interface with another molecule (referred to herein as “interface proteins”), including another protein, such as a cognate binding partner, a receptor, or an enzyme, for example. In some instances, the peptide molecules disclosed herein that mimic the functional interfaces of target molecules comprise peptide molecules comprising a linking moiety. In yet other instances, the peptide or polypeptide molecules disclosed herein may be combinatorial peptide molecules. In yet other instances, the peptide molecules disclosed herein may be fused or combined with other proteins or carrier molecules.

The present disclosure relates generally to peptide molecules, including polypeptide molecules, and more specifically to peptide molecules that mimic a target molecule interface, for example, surface or other portion of a nucleic acid, ribozyme, and/or protein, that interfaces or interacts with a binding partner or capture molecule. Accordingly, disclosed herein are peptide compositions and methods of developing peptide molecules that share functional and/or structural characteristics of a target molecule, including a nucleic acid, ribozyme and/or protein of interest. In some instances, the protein of interest is an interface protein. Also disclosed are peptide compositions and methods for developing such molecules without requiring detailed structural or physicochemical information.

Accordingly, provided herein are peptide compositions, referred to herein as functional interface mimics (FIMs), and methods of developing, making and using FIMs thereof, including for example methods of making FIMs, and methods for selecting an FIM. In some embodiments, the FIM captures key native energetic elements of a binding interaction between an interface protein of interest and its native binding partner, and presents those elements in a manner that optimizes at least one side of the protein binding interface. In certain embodiments, the FIM is smaller and more synthetically tractable than the full-length interface protein.

The FIMs provided herein exhibit at least one characteristic of the interface protein. In some embodiments, the FIMs exhibit at least one characteristic of a functional interface of the interface protein. For example, for certain types of protein characteristics in some embodiments, the functional interface is a surface interacting with a binding partner, but the rest of the interface protein also has an effect. For example, the rest of the protein may provide the three dimensional folding that holds the functional interface in place. Thus, in some embodiments of certain types of characteristics, an FIM presents a mimic of a functional interface, but the characteristic shared by the FIM may be best described as being shared with the interface protein as a whole. For some types of characteristics, in certain embodiments, the FIM shares at least one characteristic with the interface protein, wherein the characteristic is shared with a functional interface specifically.

In some aspects, provided herein is a functional interface mimic (FIM), comprising at least one peptide that exhibits at least one characteristic of an interface protein, and at least one linking moiety. In some embodiments, the linking moiety cross-links between at least two amino acid side chains of at least one peptide. In other embodiments, the linking moiety is a linker (e.g., a molecule connecting at least one part of a peptide to at least one other part of the same peptide, or a different peptide, or a combination thereof). In certain embodiments, the FIM comprises a linker and a cross-link. In some instances, the linking moiety links or connects at least one peptide through covalent or non-covalent means.

Including a linking moiety in the FIM may provide, for example, structural characteristics that contribute to the FIM exhibiting at least one characteristic of the interface protein. For example, in some embodiments, the FIM comprises one peptide and at least one linking moiety. In certain embodiments, this linking moiety connects two portions of the peptide to orient certain sections of the peptide in a way that mimics the interface of interest, or limits structural/conformational flexibility in a way that mimics the interface of interest, or both.

In certain embodiments, the FIM comprises at least two linking moieties, wherein each linking moiety is independently a cross-link or a linker. In some embodiments, the FIM comprises at least two peptides connected by at least one linking moiety, wherein each peptide independently exhibits at least one characteristic of the interface protein. Thus, in some embodiments, an FIM comprising at least two peptides may present a sequence-discontinuous mimic of a functional interface of interest, such as a binding interface. Due to the folding of proteins, a functional interface surface may not comprise a continuous linear sequence. Instead, a linear sequence may fold such that portions of the FIMs form the functional interface surface, and further, these portions may not be aligned in three dimensional space the same way they are in the linear sequence of the target molecule. The FIMs provided herein, in some embodiments, incorporate different functional and structural aspects of a functional interface surface, resulting in molecules that may have high functional similarity to the target, but with greater specificity than mimotope approaches. Thus, in some embodiments, rather than approximating the linear sequence of the interface protein of interest, the functionality of an FIM comprising at least two peptides arises from mimicking the functional interface surface itself. In other embodiments, an FIM comprises two peptides that are contiguous in the native target protein or target molecule, but are not contiguous in the FIM.

Thus, in some embodiments, provided herein is an FIM, wherein the FIM exhibits at least one characteristic of a functional interface of, for example, an interface protein or target molecule. In certain embodiments, an interface protein comprises one functional interface. In other embodiments, an interface protein comprises a plurality of functional interfaces. For example, in some embodiments, an interface protein comprises a plurality of functional interfaces, wherein each functional interface is an epitope.

Further provided herein are methods of selecting an FIM. In some embodiments, these methods comprise screening a peptide library for binding to a protein or fragment thereof, for example, the cognate binding partner of the interface protein. At least a portion of these peptides are selected based on binding with the protein or fragment thereof, and the peptides are used to generate a combinatorial library comprising FIM candidates. In some embodiments, at least a portion of the FIM candidates comprise at least two of the selected peptides connected by at least one linking moiety. In some embodiments, diversity in the combinatorial library can be increased, for example, by varying the number and identity of the peptides selected from the peptide library, and/or varying the number and identity of the linking moieties. This combinatorial library is then screened with a protein or fragment thereof (for example, the same protein or fragment thereof used to screen the peptide library), and an FIM is then selected based on binding. In some embodiments, the components of the FIM are further adjusted based on one or more additional desirable characteristics. For example, in certain embodiments, a linking moiety of a selected FIM is changed to impart desirable pharmacokinetic parameters.

In other embodiments, the methods of selecting an FIM comprise iterative optimization of a design using molecular dynamics to simulate and determine flexibility and overall stability, until the desired levels are achieved.

In some embodiments, the FIM is an immunogen. In certain embodiments, an FIM immunogen is used to produce an antibody to the FIM immunogen in, for example, an animal system (e.g., a rabbit, a mouse). Because the FIM mimics at least a portion of the interface of a target molecule, for example the surface of an interface protein, in some embodiments, an antibody produced using an FIM is expected to bind to the, for example, interface protein or target molecule. Thus, provided herein are methods of generating an antibody specific for a target by injecting into an animal an FIM, wherein the target is an interface protein.

Functional Interface Mimic

Provided herein is a functional interface mimic (FIM), comprising at least one peptide that exhibits at least one characteristic of an interface protein. In some instances, the FIM comprises at least one linking moiety. In some embodiments, the FIM comprises at least two peptides connected by at least one linking moiety, wherein the FIM shares one or more characteristics with an interface protein. In some embodiments, each peptide of the FIM independently exhibits at least one characteristic of an interface protein. In some embodiments, each linking moiety is independently a cross-link or a linker.

a. FIM Characteristics

In some embodiments, the FIMs provided herein include one or more characteristics in common with a target molecule, for example, a protein, such as an interface protein, a nucleic acid or ribozyme. Such characteristics may include, for example, structural or functional metrics, or combinations thereof. For example, in some embodiments, the FIM shares one or more structural similarities, has similar conformational entropy, shares one or more chemical descriptor similarities, shares one or more functional binding similarities, or shares one or more phenotypic similarities, or any combinations thereof, with a target molecule. In certain embodiments, the FIM shares one or more of these characteristics with a functional interface of a target molecule.

For example, the FIMs provided herein have one or more characteristics in common with a target molecule, for example an interface protein. Such characteristics may include, for example, structural or functional metrics, or combinations thereof. For example, in some embodiments, the FIM shares one or more structural similarities, has similar conformational entropy, shares one or more chemical descriptor similarities, shares one or more functional binding similarities, or shares one or more phenotypic similarities, or any combinations thereof, with an interface protein. In certain embodiments, the FIM shares one or more of these characteristics with a functional interface of an interface protein.

In some embodiments, the FIM shares one or more structural similarities with a target molecule, for example, an interface protein. In certain embodiments, a functional interface of the interface protein has one or more structural similarities in common with the FIM. In some embodiments, the structural similarity is evaluated by backbone root-mean-square deviation (RMSD) or side-chain RMSD. RMSD evaluates the average distance between atoms, and can be applied to three-dimensional structures to compare how similar two separate structures are in three-dimensional space. In some embodiments, the RMSD of the backbone, or amino acid side chains, or both, between the FIM and interface protein is lower than the RMSD between the interface protein and a different molecule (such as another FIM candidate). In certain embodiments, the RMSD of the backbone, or amino acid side chains, or both, between the FIM and a functional interface of the interface protein is lower than the RMSD between the functional interface and a different molecule (such as another FIM candidate). In some embodiments, it is a portion of the functional interface or a portion of the interface protein that is compared with the FIM. The RMSD may be evaluated, for example, using the experimentally measured structure or the simulated structure of the FIM; and the experimentally measured structure or the simulated structure of the interface protein. In some embodiments, the experimentally measured structure or the simulated structure of a functional interface of the interface protein is used. In some embodiments, an FIM is considered structurally similar to the interface protein if the backbone of the FIM has an average RMSD less than or equal to 5.0 Å, 6.0 Å, 7.0 Å, or 8.0 Å relative to the backbone of a known x-ray structure of the interface protein.

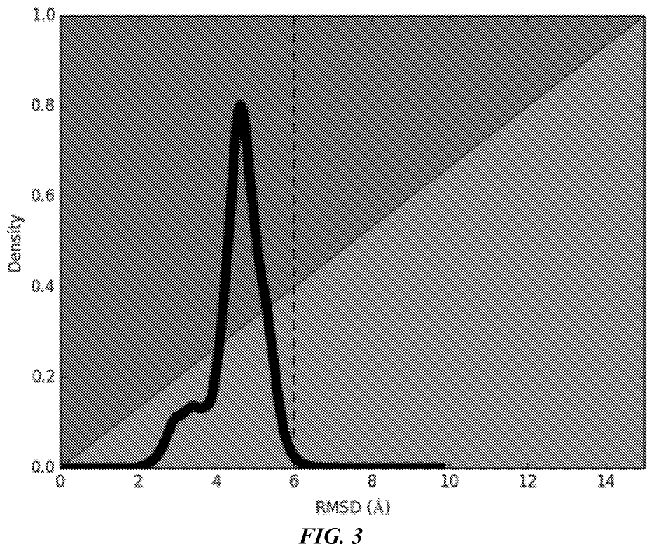

In some embodiments, the FIM has similar conformational entropy to a target molecule, for example an interface protein. In some embodiments, the conformational entropy of the FIM is similar to the conformational entropy of a functional interface of an interface protein. This conformational entropy may be evaluated, for example, using the experimentally measured structure or the simulated structure of the FIM, and the experimentally measured structure or the molecular dynamics simulated motion of the interface protein. In some embodiments, the experimentally measured structure or the molecular dynamics simulated motion of a functional interface of the interface protein is used. In certain embodiments, the conformational entropy is considered similar if the FIM molecular dynamics ensemble run under standard physiological conditions has all states with all non-hydrogen atomic position RMSDs≤6.0 Å, e.g., 5.0 Å, 6.0 Å, and in some embodiments 7.0 Å, or 8.0 Å relative to a known x-ray crystal structure of the interface protein. In some embodiments, the conformational entropy is considered similar if the FIM molecular dynamics ensemble run under standard physiological conditions has all states with all non-hydrogen atomic position RMSDs≤6.0 Å, e.g., 5.0 Å, 6.0 Å, and in some embodiments 7.0 Å, or 8.0 Å relative to a known x-ray crystal structure of a functional interface of the interface protein. Provided in FIG. 3 is an exemplary molecular dynamics simulation of FIM structural flexibility relative to a target interface crystal structure. In this figure, the FIM is considered stable and similar if the non-hydrogen atom positions of the FIM are less than 6.0 Å, e.g., 5.0 Å, 6.0 Å, and in some embodiments 7.0 Å, or 8.0 Å compared to the target interface.

In still other embodiments, the FIM has one or more chemical descriptors similar to, for example, an interface protein. In certain embodiments, the FIM has one or more chemical descriptors similar to a functional interface of the interface protein. In some embodiments, the FIM has one or more chemical descriptors that are complementary to the descriptors of a binding partner of the interface protein. Chemical descriptors may include, for example, hydrophobicity patterns, H-bonding patterns, atomic volume/radii, charge patterns, or atomic occupancy patterns, or any combinations thereof. Thus, in some embodiments, the FIM has one or more hydrophobicity patterns, H-bonding patterns, atomic volume/radii, charge patterns, or atomic occupancy patterns, or any combinations thereof, similar to that of the interface protein. In certain embodiments, the FIM has one or more hydrophobicity patterns, H-bonding patterns, atomic volume/radii, charge patterns, or atomic occupancy patterns, or any combinations thereof, similar to that of a functional interface of the interface protein. In some embodiments, the similarity is, for example, having the same chemical descriptor in common, such as one or more of the same hydrophobicity patterns, H-bonding patterns, atomic volume/radii, charge patterns, or atomic occupancy patterns. In certain embodiments, the FIM has one or more hydrophobicity patterns, H-bonding patterns, atomic volume/radii, charge patterns, or atomic occupancy patterns that are complementary to that of a binding partner of the interface protein. For example, an FIM may, in some embodiments, have a positive charge pattern that complements the negative charge pattern of a binding partner of the interface protein. These chemical descriptors may, in some embodiments, be evaluated using the experimentally measured structure or the simulated structure of the FIM, and the experimentally measured structure or the simulated structure of the interface protein, or an FIM target interface docking simulation. In some embodiments, the experimentally measured structure or the simulated structure of a functional interface of the interface protein is used.

In some embodiments, the FIM has similar functional binding as, for example, an interface protein. For example, in some embodiments, the FIM has binding with the cognate binding partner of the interface protein that is similar to the binding of the interface protein with the cognate binding partner. The cognate binding partner may be, for example, the native binding partner of an interface protein, a fragment of a native binding partner, or a modified native binding partner or fragment thereof. In some embodiments, the cognate binding partner binds under certain circumstances but not others. For example, in some embodiments, the cognate binding partner binds with the interface protein under pathological conditions. In other embodiments, the cognate binding partner binds with the interface protein under non-pathological conditions. In some embodiments, the cognate binding partner is constitutively expressed. In other embodiments, the cognate binding partner is the product of a facultative gene. In some embodiments, the cognate binding partner comprises a protein, or a fragment thereof. In certain embodiments, the cognate binding partner is a fragment of the native binding partner, or is a modified native binding partner. Modifications may include, in some embodiments, labeling of the cognate binding partner, comprising for example a fusion protein comprising at least a fragment of the cognate binding partner, with for example, a chromophore, a fluorophore, biotin, a His-tag, or combinations thereof.

For example, in some embodiments, the FIM has binding with the cognate binding partner of the interface protein that is within about two orders of magnitude, or within about one order of magnitude, of the binding of the interface protein with the cognate binding partner. In some embodiments, the similarity of binding is evaluated by comparing the binding constant (K_{d}), or the inhibitory constant (K), or the binding on-rate, or the binding off-rate, or the binding affinity of the binding pairs, or the Gibbs free energy of binding (AG).

For example, in some embodiments, the binding constant (K_{d}) of the FIM with the cognate binding partner of the interface protein is within 100-fold, within 90-fold, within 80-fold, within 70-fold, within 60-fold, within 50-fold, within 40-fold, within 30-fold, within 20-fold, within 15-fold, within 10-fold, within 8-fold, within 6-fold, within 4-fold, within 2-fold, within 1.5-fold, within 1.2-fold, or about the same as the K_{d} of the interface protein and the cognate binding partner. In other embodiments, the inhibitory constant (K) of the FIM with the cognate binding partner of the interface protein is within 100-fold, within 90-fold, within 80-fold, within 70-fold, within 60-fold, within 50-fold, within 40-fold, within 30-fold, within 20-fold, within 15-fold, within 10-fold, within 8-fold, within 6-fold, within 4-fold, within 2-fold, within 1.5-fold, within 1.2-fold, or about the same as the Kᵢ of the interface protein and the cognate binding partner. In still further embodiments, the binding on-rate of the FIM with the cognate binding partner of the interface protein is similar to the binding on-rate of the interface protein and the cognate binding partner. In some embodiments, the binding on-rate of the FIM with the cognate binding partner of the interface protein is within 100-fold, within 90-fold, within 80-fold, within 70-fold, within 60-fold, within 50-fold, within 40-fold, within 30-fold, within 20-fold, within 15-fold, within 10-fold, within 8-fold, within 6-fold, within 4-fold, within 2-fold, within 1.5-fold, within 1.2-fold, or about the same as the on-rate of the interface protein and the cognate binding partner. In other embodiments, the binding off-rate of the FIM with the cognate binding partner of the interface protein is similar to the binding off-rate of the interface protein and the cognate binding partner. In some embodiments, the binding off-rate of the FIM with the cognate binding partner of the interface protein is within 100-fold, within 90-fold, within 80-fold, within 70-fold, within 60-fold, within 50-fold, within 40-fold, within 30-fold, within 20-fold, within 15-fold, within 10-fold, within 8-fold, within 6-fold, within 4-fold, within 2-fold, within 1.5-fold, within 1.2-fold, or about the same as the off-rate of the interface protein and the cognate binding partner. In still further embodiments, the binding affinity of the FIM with the cognate binding partner of the interface protein is similar to the binding affinity of the interface protein and the cognate binding partner. In some embodiments, the binding affinity of the FIM with the cognate binding partner of the interface protein is within 100-fold, within 90-fold, within 80-fold, within 70-fold, within 60-fold, within 50-fold, within 40-fold, within 30-fold, within 20-fold, within 15-fold, within 10-fold, within 8-fold, within 6-fold, within 4-fold, within 2-fold, within 1.5-fold, within 1.2-fold, or about the same as the binding affinity of the interface protein and the cognate binding partner. In still other embodiments, the Gibbs free energy of binding of the FIM with the cognate binding partner of the interface protein is similar to the Gibbs free energy of binding of the interface protein and the cognate binding partner. In some embodiments, the Gibbs free energy of binding of the FIM with the cognate binding partner of the interface protein is within 100-fold, within 90-fold, within 80-fold, within 70-fold, within 60-fold, within 50-fold, within 40-fold, within 30-fold, within 20-fold, within 15-fold, within 10-fold, within 8-fold, within 6-fold, within 4-fold, within 2-fold, within 1.5-fold, within 1.2-fold, or about the same as the Gibbs free energy of binding of the interface protein and the cognate binding partner. In some embodiments, the FIM binds two or more different cognate binding partners of the interface protein. In certain embodiments, FIM independently shares binding similarity with each of the two or more different cognate binding partners.

In still further embodiments the FIM has phenotypic similarity to, for example an interface protein. For example, in some embodiments, the FIM has a similar in vitro or in vivo phenotype as a functional interface of the interface protein. The phenotype may include, for example, triggering or attenuating a metabolic pathway, cell signaling, apoptosis, gene expression, enzyme pathway, or cell-cycle progression.

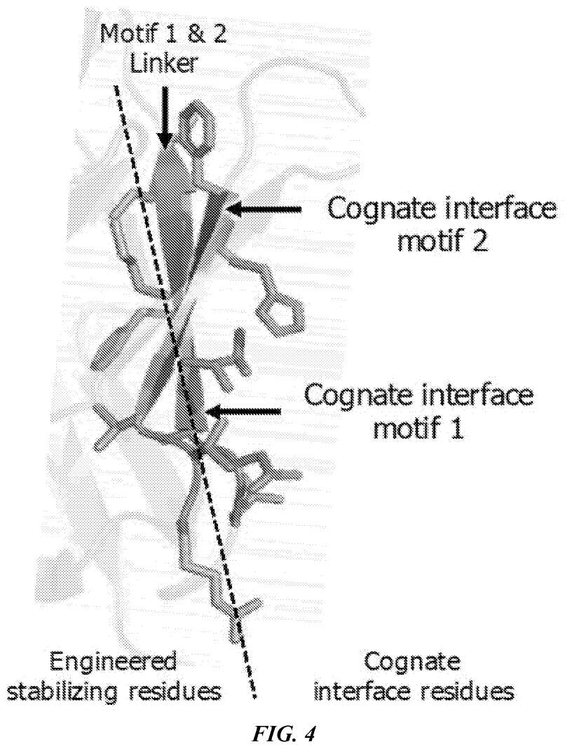

In yet other embodiments, the FIM shares sequence similarity with, for example an interface protein. In certain embodiments, the FIM shares sequence similarity with a functional interface, or a portion thereof, of the interface protein. In certain embodiments, the similarity is compared to the continuous amino acid sequence of the interface protein. In other embodiments, the sequence similarity is compared to a discontinuous sequence of the interface protein. For example, in certain embodiments, a functional interface surface of a folded interface protein is formed by discontinuous amino acid sequences, and the FIM has sequence similarity with at least a portion of the discontinuous sequences that form the surface. In other embodiments, the FIM has sequence similarity with at least a portion of a continuous amino acid sequence that forms a functional interface surface of the interface protein. For example, FIG. 4 is an exemplary FIM that demonstrates similarity to the discontinuous sequence of a target functional interface of an interface protein. This FIM also comprises amino acid residues that promote structural stability of the FIM.

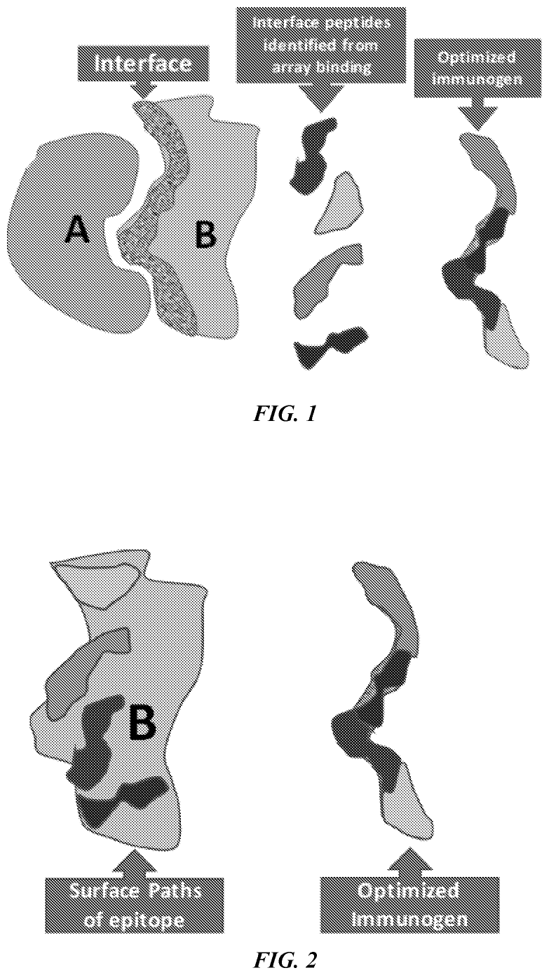

In still further embodiments, the sequence similarity is compared to a contiguous portion of a functional interface of, for example, the interface protein. In still other embodiments, the sequence similarity is compared to a discontiguous portion of a functional interface of the interface protein. For example, a functional interface of the interface protein may, in some embodiments, be formed by continuous or discontinuous sequences that, when folded in three dimensional space, form a contiguous functional interface. In some embodiments, the FIM shares sequence similarity with at least a portion of this contiguous functional interface. Provided in FIG. 1 is a diagram illustrating the development of one exemplary embodiment of an FIM, wherein the FIM shares sequence similarity with at least a portion of a contiguous functional interface. As shown in this figure, the functional protein B has a functional interface that interacts with the binding partner A. Using the methods of selecting an FIM as described herein, in some embodiments peptides are identified from a peptide library, and those peptides have sequence similarity with the functional interface. In some embodiments, these peptides are connected with linking moieties (such as linkers or cross-links) to form an FIM (selected by the methods described herein), wherein the FIM itself has a similar sequence as the contiguous functional interface of the interface protein. In other embodiments, the FIM shares sequence similarity with portions of the contiguous functional interface, wherein the portions themselves do not contact each other (e.g., discontiguous portions). Provided in FIG. 2 is a diagram illustrating the development of one exemplary embodiment of an FIM, wherein the FIM shares sequence similarity with discontiguous portions of a functional interface.

In certain embodiments, sequence similarity of the FIM and, for example, the interface protein (such as a functional interface) is evaluated using the peptide portion(s) of the FIM, not including the linkers, if present. In certain embodiments, one or more linking moieties are considered as well, for example if the FIM comprises or more linkers that comprise an amino acid. Thus, in some embodiments, the FIM has a sequence that is at least 40% identical, at least 45% identical, at least 50% identical, at least 55% identical, at least 60% identical, at least 65% identical, at least 70% identical, at least 75% identical, at least 80% identical, at least 85% identical, or at least 90% identical, to a portion of the continuous sequence of the interface protein, for example a continuous sequence that forms a functional interface. In certain embodiments, the FIM has a sequence that is at least 40% identical, at least 45% identical, at least 50% identical, at least 55% identical, at least 60% identical, at least 65% identical, at least 70% identical, at least 75% identical, at least 80% identical, at least 85% identical, or at least 90% identical, to a portion of the discontinuous sequence of the interface protein, for example the discontinuous sequence that forms a functional interface. In certain embodiments, the FIM has a sequence that is at least 40% identical, at least 45% identical, at least 50% identical, at least 55% identical, at least 60% identical, at least 65% identical, at least 70% identical, at least 75% identical, at least 80% identical, at least 85% identical, or at least 90% identical, to a contiguous portion of a functional interface of the interface protein. In still further embodiments, the FIM has a sequence that is at least 40% identical, at least 45% identical, at least 50% identical, at least 55% identical, at least 60% identical, at least 65% identical, at least 70% identical, at least 75% identical, at least 80% identical, at least 85% identical, or at least 90% identical, to two or more discontiguous portions of a functional interface of the interface protein.

In certain embodiments, conservative substitution of residue(s) in an FIM are made such that functional characteristics are maintained in the FIM as compared to the target molecule, for example, an interface protein. Accordingly, in some embodiments, the FIM has a sequence that is at least 40% similar, at least 45% similar, at least 50% similar, at least 55% similar, at least 60% similar, at least 65% similar, at least 70% similar, at least 75% similar, at least 80% similar, at least 85% similar, or at least 90% similar, to a portion of the continuous sequence of the interface protein, for example a continuous sequence that forms a functional interface. In certain embodiments, the FIM has a sequence that is at least 40% similar, at least 45% similar, at least 50% similar, at least 55% similar, at least 60% similar, at least 65% similar, at least 70% similar, at least 75% similar, at least 80% similar, at least 85% similar, or at least 90% similar, to a portion of the discontinuous sequence of the interface protein, for example the discontinuous sequence that forms a functional interface. In certain embodiments, the FIM has a sequence that is at least 40% similar, at least 45% similar, at least 50% similar, at least 55% similar, at least 60% similar, at least 65% similar, at least 70% similar, at least 75% similar, at least 80% similar, at least 85% similar, or at least 90% similar, to a contiguous portion of a functional interface of the interface protein. In still further embodiments, the FIM has a sequence that is at least 40% similar, at least 45% similar, at least 50% similar, at least 55% similar, at least 60% similar, at least 65% similar, at least 70% similar, at least 75% similar, at least 80% similar, at least 85% similar, or at least 90% similar, to two or more discontiguous portions of a functional interface of the interface protein.

b. Peptide Characteristics

The FIMs provided herein comprise at least one peptide that mimic a functional and/or structural characteristic of the target molecule, including, for example, an interface protein. In some instances, the FIMs provided herein further comprise at least one linking moiety. In some embodiments, the FIM comprises at least two peptides connected by at least one linking moiety, wherein each peptide independently exhibits at least one characteristic of an interface protein. In some embodiments, the linking moiety is a linker. Further provided herein is a population of FIM candidates, wherein a plurality of the candidates each independently comprise at least one peptide and at least one linking moiety, wherein each peptide independently exhibits at least one characteristic of the interface protein. In still further embodiments, provided herein is a population of FIM candidates, wherein a plurality of the candidates each independently comprise at least two peptides and at least one linking moiety, wherein each peptide independently exhibits at least one characteristic of the interface protein. In some embodiments, the linking moiety is a linker.

In some embodiments, each peptide of the FIM or FIM candidate independently exhibits at least one characteristic of a functional interface of a target molecule, for example, an interface protein. Such characteristics may include, for example, structural or functional metrics, or combinations thereof. For example, in some embodiments, each peptide independently shares one or more structural similarities, has similar conformational entropy, shares one or more chemical descriptor similarities, shares one or more functional binding similarities, or shares one or more phenotypic similarities, or any combinations thereof, with the interface protein. In certain embodiments, each peptide shares one or more of these characteristics with a functional interface of the interface protein.

In some embodiments, each peptide independently exhibits one to ten, one to nine, one to eight, one to seven, one to six, one to five, one to four, one to three, or one or two characteristics of, for example, an interface protein. In certain embodiments, each peptide of the FIM or FIM candidate independently shares a different characteristic with, for example, an interface protein. In other embodiments, at least two peptides of the FIM or FIM candidate share one or more of the same characteristics with, for example, an interface protein. In some embodiments, these characteristics are shared with, for example, a functional interface of an interface protein.

In some embodiments, a peptide of the FIM or FIM candidate shares one or more structural similarities with, for example, an interface protein. In certain embodiments, it is a functional interface of the interface protein that has one or more structural similarities in common with a peptide of the FIM, or with a peptide of an FIM candidate. In some embodiments, the structural similarity is evaluated by backbone RMSD or side-chain RMSD. In some embodiments, the RMSD of the backbone, or amino acid side chains, or both, between a peptide of the FIM or FIM candidate and the interface protein is lower than the RMSD between the interface protein and a different molecule (such as a different peptide). In certain embodiments, the RMSD of the backbone, or amino acid side chains, or both, between a peptide of the FIM or FIM candidate and a functional interface of the interface protein is lower than the RMSD between the interface and a different molecule (such as a different peptide). In some embodiments, it is a portion of a functional interface or a portion of the interface protein that is compared with the peptide. The RMSD may be evaluated, for example, using the experimentally measured structure or the simulated structure of the peptide of the FIM or FIM candidate; and the experimentally measured structure or the simulated structure of the interface protein. In some embodiments, the experimentally measured structure or the simulated structure of a functional interface of the interface protein is used. In some embodiments, a peptide of the FIM or FIM candidate is considered structurally similar to the interface protein if the backbone of the peptide has an average RMSD less than or equal to 6.0 Å, e.g., 5.0 Å, 6.0 Å, and in some embodiments 7.0 Å, or 8.0 Å relative to the backbone of a known x-ray structure of the interface protein.

In some embodiments, a peptide of the FIM, or a peptide of an FIM candidate, has similar conformational entropy to, for example, an interface protein. In certain embodiments, it is a functional interface of the interface protein that has similar conformational entropy to a peptide of the FIM, or to a peptide of an FIM candidate. This conformational entropy may be evaluated, for example, using the experimentally measured structure or the molecular dynamics simulated motion of the peptide, and the experimentally measured structure or the simulated structure of the interface protein. In some embodiments, the experimentally measured structure or the simulated structure of a functional interface of the interface protein is used. In some embodiments, the conformational entropy is considered similar if a peptide molecular dynamics ensemble run under standard physiological conditions has all states with all non-hydrogen atomic portions RMSDs≤6.0 Å, e.g., 5.0 Å, 6.0 Å, and in some embodiments 7.0 Å, or 8.0 Å relative to a known x-ray crystal structure of the interface protein. In certain embodiments, the conformational entropy is considered similar if a peptide molecular dynamics ensemble run under standard physiological conditions has all states with all non-hydrogen atomic portions RMSDs≤6.0 Å, e.g., 5.0 Å, 6.0 Å, and in some embodiments 7.0 Å, or 8.0 Å relative to a known x-ray crystal structure of a functional interface of the interface protein.

In still other embodiments, a peptide of the FIM has similar chemical descriptors as, for example, an interface protein. In certain embodiments, a peptide of the FIM has similar chemical descriptors as a functional interface of the interface protein. In still other embodiments, a peptide of an FIM candidate has similar chemical descriptors as the interface protein, or similar chemical descriptors as a functional interface of the interface protein. In some embodiments, a peptide of an FIM or FIM candidate has one or more chemical descriptors that are complementary to the descriptors of a binding partner of the interface protein. Chemical descriptors may include, for example, hydrophobicity patterns, H-bonding patterns, atomic volume/radii, charge patterns, or atomic occupancy patterns, or any combinations thereof. Thus, in some embodiments, a peptide of the FIM or FIM candidate has one or more hydrophobicity patterns, H-bonding patterns, atomic volume/radii, charge patterns, or atomic occupancy patterns, or any combinations thereof, similar to that of the interface protein. In certain embodiments, the peptide has one or more hydrophobicity patterns, H-bonding patterns, atomic volume/radii, charge patterns, or atomic occupancy patterns, or any combinations thereof, similar to that of a functional interface of the interface protein. In some embodiments, the similarity is, for example, having the same chemical descriptor in common, such as one or more of the same hydrophobicity patterns, H-bonding patterns, atomic volume/radii, charge patterns, or atomic occupancy patterns. In certain embodiments, the peptide has one or more hydrophobicity patterns, H-bonding patterns, atomic volume/radii, charge patterns, or atomic occupancy patterns that are complementary to that of a binding partner of the interface protein. For example, a peptide may, in some embodiments, have a positive charge pattern that complements the negative charge pattern of a binding partner of the interface protein. These chemical descriptors may, in some embodiments, be evaluated using an experimentally measured structure or a simulated structure of the peptide, and an experimentally measured structure or a simulated structure of the interface protein, or an FIM target interface docking simulation. In some embodiments, an experimentally measured structure or a simulated structure of a functional interface of the interface protein is used.

For example, in some embodiments, a peptide of the FIM, or a peptide of an FIM candidate, has binding with the cognate binding partner of the interface protein that is similar to the binding of the interface protein with the cognate binding partner. The cognate binding partner may be, for example, the native binding partner, a fragment of a native binding partner, or a modified native binding partner or fragment thereof. In some embodiments, the cognate binding partner binds under certain circumstances but not others. For example, in some embodiments, the cognate binding partner binds with the interface protein under pathological conditions. In other embodiments, the cognate binding partner binds with the interface protein under non-pathological conditions. In some embodiments, the cognate binding partner is constitutively expressed. In other embodiments, the cognate binding partner is the product of a facultative gene. In some embodiments, the cognate binding partner comprises a protein, or a fragment thereof. In certain embodiments, the cognate binding partner is a fragment of the native binding partner, or is a modified native binding partner. Modifications may include, in some embodiments, a fusion protein comprising at least a fragment of the native binding partner; labeling with a chromophore; labeling with a fluorophore; labeling with biotin; or labeling with a His-tag.

For example, in some embodiments, a peptide of the FIM or FIM candidate has binding with the cognate binding partner of the interface protein that is within about two orders of magnitude, or within about one order of magnitude, of the binding of the interface protein with the cognate binding partner. In some embodiments, the similarity of binding is evaluated by comparing the binding constant (K_{d}), or the inhibitory constant (K), or the binding on-rate, or the binding off-rate, or the binding affinity of the binding pairs, or the Gibbs free energy of binding (AG).

For example, in some embodiments, the binding constant (K_{d}) of the peptide with the cognate binding partner of the interface protein is within 1000-fold, within 800-fold, within 600-fold, within 400-fold, within 200-fold, within 100-fold, within 90-fold, within 80-fold, within 70-fold, within 60-fold, within 50-fold, within 40-fold, within 30-fold, within 20-fold, within 10-fold, within 8-fold, within 6-fold, within 4-fold, within 2-fold, within 1.5-fold, within 1.2-fold, or about the same as the K_{d} of the interface protein and the cognate binding partner. In other embodiments, the inhibitory constant (K) of a peptide of the FIM with the cognate binding partner of the interface protein is within 1000-fold, within 800-fold, within 600-fold, within 400-fold, within 200-fold, within 100-fold, within 90-fold, within 80-fold, within 70-fold, within 60-fold, within 50-fold, within 40-fold, within 30-fold, within 20-fold, within 10-fold, within 8-fold, within 6-fold, within 4-fold, within 2-fold, within 1.5-fold, within 1.2-fold, or about the same as the Kᵢ of the interface protein and the cognate binding partner. In still further embodiments, the binding on-rate of the peptide with the cognate binding partner of the interface protein is similar to the binding on-rate of the interface protein and the cognate binding partner. In some embodiments, the binding on-rate of the peptide with the cognate binding partner of the interface protein is within 1000-fold, within 800-fold, within 600-fold, within 400-fold, within 200-fold, within 100-fold, within 90-fold, within 80-fold, within 70-fold, within 60-fold, within 50-fold, within 40-fold, within 30-fold, within 20-fold, within 10-fold, within 8-fold, within 6-fold, within 4-fold, within 2-fold, within 1.5-fold, within 1.2-fold, or about the same as the on-rate of the interface protein and the cognate binding partner. In other embodiments, the binding off-rate of the peptide with the cognate binding partner of the interface protein is similar to the binding off-rate of the interface protein and the cognate binding partner. In some embodiments, the binding off-rate of the peptide with the cognate binding partner of the interface protein is within 1000-fold, within 800-fold, within 600-fold, within 400-fold, within 200-fold, within 100-fold, within 90-fold, within 80-fold, within 70-fold, within 60-fold, within 50-fold, within 40-fold, within 30-fold, within 20-fold, within 10-fold, within 8-fold, within 6-fold, within 4-fold, within 2-fold, within 1.5-fold, within 1.2-fold, or about the same as the off-rate of the interface protein and the cognate binding partner. In still further embodiments, the binding affinity of the peptide with the cognate binding partner of the interface protein is similar to the binding affinity of the interface protein and the cognate binding partner. In some embodiments, the binding affinity of the peptide with the cognate binding partner of the interface protein is within 1000-fold, within 800-fold, within 600-fold, within 400-fold, within 200-fold, within 100-fold, within 90-fold, within 80-fold, within 70-fold, within 60-fold, within 50-fold, within 40-fold, within 30-fold, within 20-fold, within 10-fold, within 8-fold, within 6-fold, within 4-fold, within 2-fold, within 1.5-fold, within 1.2-fold, or about the same as the binding affinity of the interface protein and the cognate binding partner. In some embodiments, the Gibbs free energy of binding of the peptide with the cognate binding partner of the interface protein is within 1000-fold, within 800-fold, within 600-fold, within 400-fold, within 200-fold, within 100-fold, within 90-fold, within 80-fold, within 70-fold, within 60-fold, within 50-fold, within 40-fold, within 30-fold, within 20-fold, within 10-fold, within 8-fold, within 6-fold, within 4-fold, within 2-fold, within 1.5-fold, within 1.2-fold, or about the same as the Gibbs free energy of binding of the interface protein and the cognate binding partner. In some embodiments, the peptide binds to two or more different cognate binding partners of the interface protein. In some embodiments, the peptide independently shares binding similarity with each of the two or more different cognate binding partners of the interface protein.

In yet other embodiments, at least one of the characteristics shared with, for example, an interface protein by at least one of the peptides is sequence similarity. In certain embodiments, the similarity is compared to the continuous amino acid sequence of the interface protein. In other embodiments, the sequence similarity is compared to a discontinuous sequence of the interface protein. For example, in certain embodiments, a functional interface surface of a folded interface protein is formed by discontinuous amino acid sequences, and at least one peptide of the FIM has sequence similarity with at least a portion of the discontinuous sequences that form the surface. In some embodiments, at least one peptide of the FIM or FIM candidate has sequence similarity with at least a portion of a continuous amino acid sequence that forms a functional interface of the interface protein. In some embodiments, at least one peptide of the FIM or FIM candidate has a sequence that is at least 50% identical, at least 55% identical, at least 60% identical, at least 65% identical, at least 70% identical, at least 75% identical, at least 80% identical, at least 85% identical, at least 90% identical, at least 95% identical, or at least 99% identical to at least a portion of a continuous sequence of the interface protein, such as a continuous sequence that forms a functional interface. In certain embodiments, at least one peptide of the FIM or FIM candidate has a sequence that is at least 40% identical, at least 45% identical, at least 50% identical, at least 55% identical, at least 60% identical, at least 65% identical, at least 70% identical, at least 75% identical, at least 80% identical, at least 85% identical, or at least 90% identical, to at least a portion of the discontinuous sequence of the interface protein, for example the discontinuous sequence that forms a functional interface. In certain embodiments, at least one peptide of the FIM or FIM candidate has a sequence that is at least 40% identical, at least 45% identical, at least 50% identical, at least 55% identical, at least 60% identical, at least 65% identical, at least 70% identical, at least 75% identical, at least 80% identical, at least 85% identical, or at least 90% identical, to a contiguous portion of a functional interface of the interface protein. In still further embodiments, at least one peptide of the FIM or FIM candidate has a sequence that is at least 40% identical, at least 45% identical, at least 50% identical, at least 55% identical, at least 60% identical, at least 65% identical, at least 70% identical, at least 75% identical, at least 80% identical, at least 85% identical, or at least 90% identical, to two or more discontiguous portions of a functional interface of the interface protein. In some embodiments, for FIMs or FIM candidates that comprise at least two peptides, two or more peptides of the FIM or FIM candidate independently share sequence similarity with the interface protein, such as with a functional interface of the interface protein.

In certain embodiments, conservative substitution of residue(s) in an FIM are made such that functional characteristics are maintained in the FIM as compared to the target molecule, for example, an interface protein. Accordingly, in some embodiments, the FIM has a sequence that is at least 40% similar, at least 45% similar, at least 50% similar, at least 55% similar, at least 60% similar, at least 65% similar, at least 70% similar, at least 75% similar, at least 80% similar, at least 85% similar, or at least 90% similar, to a portion of the continuous sequence of the interface protein, for example a continuous sequence that forms a functional interface. In certain embodiments, the FIM has a sequence that is at least 40% similar, at least 45% similar, at least 50% similar, at least 55% similar, at least 60% similar, at least 65% similar, at least 70% similar, at least 75% similar, at least 80% similar, at least 85% similar, or at least 90% similar, to a portion of the discontinuous sequence of the interface protein, for example the discontinuous sequence that forms a functional interface. In certain embodiments, the FIM has a sequence that is at least 40% similar, at least 45% similar, at least 50% similar, at least 55% similar, at least 60% similar, at least 65% similar, at least 70% similar, at least 75% similar, at least 80% similar, at least 85% similar, or at least 90% similar, to a contiguous portion of a functional interface of the interface protein. In still further embodiments, the FIM has a sequence that is at least 40% similar, at least 45% similar, at least 50% similar, at least 55% similar, at least 60% similar, at least 65% similar, at least 70% similar, at least 75% similar, at least 80% similar, at least 85% similar, or at least 90% similar, to two or more discontiguous portions of a functional interface of the interface protein.

c. Peptide Components

The FIMs provided herein comprise at least one peptide, and in some instances, at least one linking moiety. In some embodiments, the FIMs provided herein comprise at least two peptides connected by at least one linking moiety. In some embodiments, the linking moiety is independently a cross-link or a linker. In some embodiments of the methods provided herein, at least one peptide of the FIM comprises interface residues retained from the target interface protein. In certain embodiments, each peptide of the FIM comprises interface residues retained from the target interface protein. In some embodiments of the methods of selecting an FIM as described herein, a peptide library is contacted by a protein or fragment thereof, and at least a portion of the peptides are selected based on binding for use in generating a combinatorial library of FIM candidates. In certain methods of selecting an FIM as described herein, the combinatorial library comprises FIM candidates, wherein each FIM candidate independently comprises at least two peptides connected by at least one linking moiety. FIG. 4 presents an exemplary FIM comprising two peptides connected by one linker.

In some embodiments, each peptide of the peptide library, FIM candidate, or FIM independently comprises between 2 to 100 amino acids. In some embodiments, each peptide independently comprises between 2 to 80 amino acids, between 2 to 70 amino acids, between 2 to 60 amino acids, or between 2 to 50 amino acids. In some embodiments, each peptide independently comprises between 2 to 40 amino acids. In some embodiments, each peptide independently comprises between 2 to 40 amino acids, between 2 to 30 amino acids, between 2 to 25 amino acids, between 2 to 20 amino acids, between 2 to 15 amino acids, between 5 to 30 amino acids, between 5 to 25 amino acids, between 5 to 20 amino acids, or between 5 to 15 amino acids. In some embodiments, each peptide independently comprises between 9 and 15 amino acids. In some embodiments, the amino acids may be natural or non-natural amino acids, for example, non-proteinogenic amino acids, including but not limited to peptide nucleic acids, (3-amino acids, homo-amino acids, N-methyl amino acids and/or other amino acid derivatives.

In some embodiments, the FIM comprises one peptide and at least one linking moiety. In some embodiments, the FIM, or FIM candidate, comprises between 1 to 20 peptides and at least one linking moiety. In some embodiments, the FIM, or FIM candidate, comprises between 2 to 20 peptides connected by independent linking moieties; in some embodiments the peptides of the FIM or FIM candidate are connected by at least one linking moiety. In some embodiments, each linking moiety is independently a cross-link or a linker. In certain embodiments, each linking moiety is independently a linker. In certain embodiments, the FIM or FIM candidate comprises between 2 to 20 peptides, between 2 to 18 peptides, between 2 to 16 peptides, or between 2 to 14 peptides. In certain embodiments, the FIM or FIM candidate comprises between 2 to 12 peptides, between 2 to 11 peptides, between 2 to 10 peptides, between 2 to 9 peptides, between 2 to 8 peptides, between 2 to 7 peptides, between 2 to 6 peptides, between 2 to 5 peptides, between 2 to 4 peptides, between 3 to 12 peptides, between 3 to 11 peptides, between 3 to 10 peptides, between 3 to 9 peptides, between 3 to 8 peptides, between 3 to 7 peptides, between 3 to 6 peptides, or between 3 to 5 peptides. In some embodiments, the FIM comprises 4 peptides. In some embodiments, the FIM comprises 12 or fewer peptides. In some embodiments, an FIM candidate comprises 4 peptides. In some embodiments, each FIM candidate independently comprises 12 or fewer peptides.

In some embodiments, the FIM comprises one peptide comprising between 2 to 100 amino acids; or between 2 to 80 amino acids; or between 2 to 60 amino acids; or between 2 to 40 amino acids; between 2 to 35 amino acids; or between 2 to 30 amino acids; or between 2 to 25 amino acids; or between 2 to 20 amino acids; or between 2 to 15 amino acids; or between 5 to 30 amino acids; or between 5 to 25 amino acids; or between 5 to 20 amino acids; or between 5 to 15 amino acids; or between 9 and 15 amino acids.

In some embodiments, the FIM or FIM candidate comprises between 2 to 20 peptides, wherein each peptide independently comprises between 2 to 100 amino acids; or between 2 to 20 peptides, wherein each peptide independently comprises between 2 to 80 amino acids; or between 2 to 20 peptides, wherein each peptide independently comprises between 2 to 60 amino acids; or between 2 to 20 peptides, wherein each peptide independently comprises between 2 to 40 amino acids. In some embodiments, the FIM or FIM candidate comprises between 2 to 16 peptides, wherein each peptide independently comprises between 2 to 100 amino acids; or between 2 to 16 peptides, wherein each peptide independently comprises between 2 to 80 amino acids; or between 2 to 16 peptides, wherein each peptide independently comprises between 2 to 60 amino acids; or between 2 to 16 peptides, wherein each peptide independently comprises between 2 to 40 amino acids.

In some embodiments, the FIM comprises between 2 to 12 peptides, wherein each peptide independently comprises between 2 to 40 amino acids. In some embodiments, each FIM candidate independently comprises between 2 to 12 peptides, wherein each peptide independently comprises between 2 to 40 amino acids. In some embodiments, the FIM or an FIM candidate comprises between 2 to 12 peptides, wherein each peptide independently comprises between 2 to 35 amino acids, between 2 to 30 amino acids, between 2 to 25 amino acids, between 2 to 20 amino acids, between 2 to 15 amino acids, between 5 to 30 amino acids, between 5 to 25 amino acids, between 5 to 20 amino acids, between 5 to 15 amino acids, or between 9 and 15 amino acids. In some embodiments, the FIM or an FIM candidate comprises between 2 to 8 peptides, wherein each peptide independently comprises between 2 to 35 amino acids, between 2 to 30 amino acids, between 2 to 25 amino acids, between 2 to 20 amino acids, between 2 to 15 amino acids, between 5 to 30 amino acids, between 5 to 25 amino acids, between 5 to 20 amino acids, between 5 to 15 amino acids, or between 9 and 15 amino acids. In some embodiments, the FIM or an FIM candidate comprises 4 peptides, wherein each peptide independently comprises between 2 to 35 amino acids, between 2 to 30 amino acids, between 2 to 25 amino acids, between 2 to 20 amino acids, between 2 to 15 amino acids, between 5 to 30 amino acids, between 5 to 25 amino acids, between 5 to 20 amino acids, between 5 to 15 amino acids, or between 9 and 15 amino acids. In one embodiment, the FIM comprises 4 peptides, wherein each peptide independently comprises between 9 to 15 amino acids. In certain embodiments, an FIM candidate comprises 4 peptides, wherein each peptide independently comprises between 9 to 15 amino acids.

In some embodiments, each peptide of the peptide library, FIM candidate, or FIM independently comprises at least 4 amino acids, at least 5 amino acids, at least 6 amino acids, at least 7 amino acids, at least 8 amino acids, at least 9 amino acids, at least 10 amino acids, at least 11 amino acids, at least 12 amino acids, at least 13 amino acids, at least 14 amino acids, at least 15 amino acids, at least 16 amino acids, at least 17 amino acids, at least 18 amino acids, at least 19 amino acids, at least 20 amino acids, at least 21 amino acids, at least 22 amino acids, at least 23 amino acids, at least 24 amino acids, at least 25 amino acids, at least 26 amino acids, at least 27 amino acids, at least 28 amino acids, at least 29 amino acids, or at least 30 amino acids in length. In still other embodiments, the peptides are not more than 15 amino acids, not more than 14 amino acids, not more than 13 amino acids, not more than 12 amino acids, not more than 11 amino acids, not more than 10 amino acids, not more than 9 amino acids or not more than 8 amino acids in length. In still other embodiments, the peptides on the library have an average length of about 6 amino acids, about 7 amino acids, about 8 amino acids, about 9 amino acids, about 10 amino acids, about 11 amino acids, about 12 amino acids, about 13 amino acids, about 14 amino acids, or about 15 amino acids.

In some embodiments, the FIM or FIM candidate, including the peptides and linking moieties (such as a linker), comprises at most 4 amino acids, at most 5 amino acids, at most 6 amino acids, at most 7 amino acids, at most 8 amino acids, at most 9 amino acids, at most 10 amino acids, at most 11 amino acids, at most 12 amino acids, at most 13 amino acids, at most 14 amino acids, at most 15 amino acids, at most 16 amino acids, at most 17 amino acids, at most 18 amino acids, at most 19 amino acids, at most 20 amino acids, at most 21 amino acids, at most 22 amino acids, at most 23 amino acids, at most 24 amino acids, at most 25 amino acids, at most 26 amino acids, at most 27 amino acids, at most 28 amino acids, at most 29 amino acids, at most 30 amino acids, at most 40 amino acids, at most 50 amino acids, at most 60 amino acids, at most 70 amino acids, at most 80 amino acids, at most 90 amino acids, at most 100 amino acids, at most 150 amino acids, at most 200 amino acids, at most 250 amino acids, at most 300 amino acids, at most 350 amino acids, at most 400 amino acids, at most 450 amino acids, or at most 500 amino acids. In some embodiments, the FIM, including the peptides and linking moieties (such as linkers), comprises at most 60 amino acids. In other embodiments, the FIM or FIM candidate, including the peptides and linking moieties (such as linkers), comprises at most 50 amino acids. In some embodiments, the FIM or FIM candidate, including the peptides and linking moieties (such as linkers), comprises between 4 to 500 amino acids, between 10 to 450 amino acids, between 10 to 400 amino acids, between 10 to 350 amino acids, between 10 to 300 amino acids, between 10 to 250 amino acids, between 10 to 200 amino acids, between 10 to 150 amino acids, between 10 to 100 amino acids, between 30 to 70 amino acids, or between 36 to 60 amino acids.

In certain embodiments, the FIM comprises 4 peptides each independently comprising 5 to 25 amino acids, and in some instances 9 to 15 amino acids, wherein in total the FIM comprises 50 or fewer amino acids. In some embodiments, an FIM candidate comprises 4 peptides each independently comprising 5 to 25 amino acids, and in some instances 9 to 15 amino acids, wherein in total the FIM candidate comprises 50 or fewer amino acids.

d. Linking Moieties

In some instances, the FIMs provided herein comprise at least one linking moiety. In some embodiments, the FIM comprises at least one peptide and at least one linking moiety. In certain embodiments, the FIM comprises at least two peptides connected by at least one linking moiety. In some methods of selecting an FIM as described herein, the combinatorial library comprises FIM candidates, wherein each FIM candidate independently comprises at least two peptides connected by at least one linking moiety. In some embodiments, the FIM or FIM candidate comprises more than two peptides connected by at least one linking moiety. In certain embodiments, the FIM comprises N number of peptides, and N−1 number of linking moieties. In some embodiments, the FIM candidate comprises N number of peptides, and N−1 number of linking moieties. In some embodiments, the FIM or FIM candidate comprises N number of peptides, and N number of linking moieties; or N number of peptides, and N+1 number of linking moieties; or N number of peptides, and N+2 number of linking moieties; or N number of peptides, and N−2 number of linking moieties, wherein N is 3 or larger.

In some embodiments, each linking moiety is independently a cross-link or a linker. A cross-link includes, for example, a covalent bond between the side chain of one amino acid and a moiety of another amino acid, wherein the amino acids may independently be natural or non-natural amino acids. Cross-links may include, for example, a covalent bond between the side chains of two amino acids, or between the side chain of one amino acid and the amine or carboxyl group of another amino acid. Such cross-links may form, for example, within one peptide (such as an FIM comprising one peptide and at least one linking moiety) or between two separate peptides (such as an FIM comprising at least two peptides connected by at least one linking moiety). In some embodiments, the FIM comprises a mixture of both (such as an FIM comprising at least two peptides and at least two linking moieties, wherein one linking moiety is an intra-peptide cross-link and one linking moiety is an inter-peptide cross-link). Cross-links include, for example, a disulfide bond between two thiol groups of amino acid side chains (such as cysteines), and an amide bond between an amine group and a carboxylic acid group (such as an amide forming between diaminopimelic acid and aspartic acid), wherein at least one of the amine and the carboxylic acid group is located on a side chain of an amino acid (e.g., the amide bond is not a backbone amide bond). In some embodiments, an amide cross-link is a lactam. In some embodiments, the cross-link is an oxime. In some embodiments, the cross-link is a hydrazone. In some embodiments, a cross-link comprises a covalent bond between a side chain of an amino acid and a moiety of another amino acid, wherein one or both of the side chain and the moiety are modified to form the covalent bond. Such modifications may include, for example, oxidation, reduction, reaction with a catalyst to form an intermediate, or other modifications known to one of skill in the art.

A linker includes, for example, a molecule that covalently bonds to at least two sites of a peptide, or between at least two peptides. A linker may bond to two sites within one peptide (such as an FIM comprising one peptide and at least one linking moiety) or between two separate peptides (such as an FIM comprising at least two peptides connected by at least one linking moiety), or a mixture of both (such as an FIM comprising at least two peptides and at least two linking moieties, wherein one linking moiety is an intra-peptide linker and one linking moiety is an inter-peptide linker). In FIMs or FIM candidates comprising at least two peptides and at least one linking moiety, wherein at least one of the linking moieties is a linker, the at least two peptides and at least one linker may be connected in a variety of different configurations. For example, in some embodiments, each linker in the FIM or FIM candidate has two peptide-attachment sites, there are N number of peptides and N−1 number of linkers, each site of each linker is attached to a peptide, and the FIM or FIM candidate has a peptide-linker-peptide-etc. pattern, ending with a peptide. In still further embodiments, one or more linkers has more than two peptide-attachment sites. FIMs or FIM candidates comprising at least one linker with more than two peptide-attachment sites may in some embodiments comprise a branching point, for example a linker that is independently attached to three peptides. In other embodiments, an FIM or FIM candidate comprising a linker with more than two peptide-attachment sites does not include a branching point, for example a linker with three peptide-attachment sites but which is only attached to two peptides.

In some embodiments, the FIM or FIM candidate comprises 1 linking moiety, 2 linking moieties, 3 linking moieties, 4 linking moieties, 5 linking moieties, 6 linking moieties, 7 linking moieties, 8 linking moieties, 9 linking moieties, 10 linking moieties, or 11 linking moieties. In certain embodiments, the FIM comprises between 1 to 11 linking moieties, between 1 to 10 linking moieties, between 1 to 9 linking moieties, between 1 to 8 linking moieties, between 1 to 7 linking moieties, between 1 to 6 linking moieties, between 1 to 5 linking moieties, between 1 to 4 linking moieties, between 1 to 3 linking moieties, between 2 to 11 linking moieties, between 2 to 10 linking moieties, between 2 to 9 linking moieties, between 2 to 8 linking moieties, between 2 to 7 linking moieties, between 2 to 6 linking moieties, between 2 to 5 linking moieties, between 2 to 4 linking moieties, between 3 to 11 linking moieties, between 3 to 10 linking moieties, between 3 to 9 linking moieties, between 3 to 8 linking moieties, between 3 to 7 linking moieties, between 3 to 6 linking moieties, or between 3 to 5 linking moieties. In some embodiments, the FIM comprises 1, 2, 3, 4, or 5 linking moieties. In other embodiments, the FIM comprises 1, 2, or 3 linking moieties. In some embodiments, the FIM comprises 3 linking moieties. In some embodiments, each FIM candidate independently comprises 1, 2, 3, 4, or 5 linking moieties. In other embodiments, each FIM candidate independently comprises 1, 2, or 3 linking moieties. In some embodiments, each FIM candidate independently comprises 3 linking moieties. In certain embodiments, each linking moiety is independently a linker. In other embodiments, each linking moiety is independently a cross-link. In still further embodiments, at least one linking moiety is a linker, and each remaining linking moiety is independently a linker or a cross-link. In other embodiments, at least one linking moiety is a cross-link, and each remaining linking moiety is independently a linker or a cross-link. In some embodiments, the FIM or FIM candidate comprises at least two linking moieties, wherein each linking moiety is independently a linker or a cross-link.

In some embodiments, at least one linking moiety is a linker, and at least one linker independently comprises one or more amino acids. In certain embodiments, a linker that comprises one or more amino acids is distinct from the one or more peptides of the FIM or FIM candidates because the one or more peptides are each identified via: a peptide library screen, and/or structure-based design, and/or simulations; and the linker is identified after the FIM or FIM candidate peptides have been selected, via: screening a combinatorial library that includes linker variations, and/or structure-based design, and/or simulations. In certain embodiments, the linker is a region that separates and presents FIM peptides in a structural, chemical, and dynamical manner that reflects the structure and/or function of a functional interface of the interface protein. In still further embodiments, the linker does not have a function on its own when not connected to the peptides of the FIM or FIM candidate (e.g., does not exhibit binding to a binding partner of the interface protein), while the peptides of the FIM or FIM candidate do have one or more functions (e.g., has binding with a binding partner of the interface protein that is similar to the binding of the interface protein with the binding partner). For linkers comprising one or more amino acids, the one or more amino acids may be, in some embodiments, naturally occurring amino acids or non-naturally occurring amino acids. For example, in some embodiments, a linker independently comprises one or more alpha-amino acids, one or more beta-amino acids, or one or more gamma-amino acids, or any combinations thereof. In certain embodiments, a linker independently comprises a cyclic beta residue, such as APC or ACPC. In some embodiments, a linker independently comprises one, two, three, four, five, or six amino acids. In certain embodiments, a linker independently comprises one or two amino acids. In some embodiments, a linker independently comprises Gly-Pro or Ala-Pro. In other embodiments, the linker comprises an amino acid sequence having one or more glycine residues, one or more serine residues, or one or more proline residues. For example, in certain embodiments the linker has an amino acid sequence selected from the group consisting of GSG, (GGGGS)n, (GSG)n, GGGSGGGGS, GGGGSGGGS, (PGSG)n, and PGSGSG, where n is an integer between 1 and 10. In certain embodiments, the FIM comprises at least one linker, wherein each linker does not comprise amino acids. In some embodiments, the FIM comprises at least one linker, wherein each linker does not comprise a natural amino acid. In still further embodiments, the FIM comprises at least one linker, wherein each linker comprises independently optionally comprises one or more non-natural amino acids. In some embodiments, each FIM candidate independently comprises at least one linker, wherein each linker does not comprise amino acids. In some embodiments, each FIM candidate independently comprises at least one linker, wherein each linker does not comprise a natural amino acid. In still further embodiments, each FIM candidate independently comprises at least one linker, wherein each linker independently optionally comprises one or more non-natural amino acids.

In other embodiments, at least one linker independently comprises a polymer. For example, in some embodiments, at least one linker independently comprises a polyethylene glycol (PEG). The PEG may comprise, for example, at least 3 monomer units, at least 4 monomer units, at least 5 monomer units, at least 6 monomer units, at least 7 monomer units, at least 8 monomer units, at least 9 monomer units, at least 10 monomer units, at least 11 monomer units, or at least 12 monomer units. In some embodiments, the PEG comprises between 3 to 12 monomer units, between 3 to 6 monomer units, between 6 to 12 monomer units, or between 4 to 8 monomer units. Thus, for example, in some embodiments at least one linker independently comprises PEG3 (comprising 3 monomer units), PEG6, or PEG12. In some embodiments, at least one linker is independently PEG3, PEG6, or PEG12. In certain embodiments, at least one linker independently comprises a multi-arm PEG. For example, in certain embodiments, at least one linker independently comprises a 4-arm PEG, or an 8-arm PEG. In certain embodiments, each arm independently comprises between 3 to 12 monomer units, or between 3 to 6 monomer units, or between 6 to 12 monomer units, or between 4 to 8 monomer units. In certain embodiments, each arm of the multi-arm PEG comprises the same number of monomer units, for example a 4- or 8-arm PEG wherein each arm comprises 3 monomer units, 6 monomer units, or 12 monomer units.

In some embodiments, at least one linker independently comprises a dendrimer. Dendrimers include, for example, molecules with a tree-like branching architecture, comprising a symmetric core from which molecular moieties radially extend, with branch points forming new layers in the molecule. Each new branch point introduces a new, larger layer, and these radial extensions often terminate in functional groups at the exterior terminal surface of the dendrimer. Thus, increasing the number of branch points in turn amplifies the possible number of terminal functional groups at the surface.

In some embodiments, at least one linker comprises a small molecule that is not an amino acid. For example, in some embodiments, at least one linker comprises a benzodiazepine. In some embodiments, the linker comprises the product of a sulfhydryl-maleimide reaction, such as a pyrrolidine dione moiety, for example pyrrolidine-2,5-dione moiety. In some embodiments, the linker comprises an amidine moiety. In some embodiments, the linker comprises a thioether moiety.

In some embodiments, at least one linker comprises trans-pyrrolidine-3,4-dicarboxamide.

In some embodiments, at least one linker comprises at least one nucleic acid. For example, in some embodiments, the linker comprises at least one deoxyribonucleic acid, or at least one ribonucleic acid, or a combination thereof.

In some embodiments, the inclusion of one or more linking moieties in the FIM or the FIM candidate may impart a particular structural or functional characteristic of interest, or a combination thereof. In some embodiments, one or more linking moieties is selected to introduce a structural or functional characteristic, or combination thereof. Structural characteristics may include, for example, increased structural flexibility, decreased structural flexibility, a directional feature (such as a turn, or a straight linker), increased length, or decreased length. Functional characteristics may include, for example, enhanced solubility, one or more protonation sites, one or more proteolytic sites, one or more enzymatic modification sites, one or more oxidation sites, a label, or a capture handle. Linkers may, in some embodiments, comprise one or more functional characteristics, or one or more structural characteristics, or a combinations thereof.

For example, in some embodiments, one or more linkers independently introduce a structural “turn” into the FIM, or into the FIM candidate. Examples of “turn” linker include Gly-Pro, Ala-Pro, and trans-pyrrolidine-3,4-dicarboxamide. In some embodiments, one or more linking moieties independently introduce structural flexibility into the FIM, or into the FIM candidate. For example, including in an FIM or FIM candidate a linker that is longer and/or less sterically hindered than another linker may, in some embodiments, result in the molecule having greater structural flexibility than if the other linker were used instead. In some embodiments, including in an FIM a cross-link at a particular location of one or more peptides, or between certain amino acid side chains, results in the molecule having greater structural flexibility than if the cross-link was at a different location or between different side chains (e.g., a disulfide or an amide cross-link). In other embodiments, one or more linking moieties independently decreases structural flexibility in the FIM or FIM candidate, such as including a linker that is shorter and/or more sterically hindered than another linker, or a cross-link at a location or of a type that reduces flexibility of one or more peptides.

e. Uses of FIMs

The FIMs provided herein, and identified by the methods provided herein, may be used in a variety of ways. These uses may include, for example, as a capture agent, therapeutic molecule, labeling reagent, enzyme substrate, pharmacokinetic enhancer, Fc receptor binder, drug carrier, in chimeric antigen receptor-T cell development, diagnostic reagent, or as an immunogen.

In some embodiments, the FIM is an immunogen. Immunogen FIMs can be used, in some embodiments, to produce antibodies that target the interface protein. In some embodiments, the FIM can be used to produce an anti-interface protein (e.g., a monoclonal or polyclonal antibody). Thus, in some embodiments, provided herein is an antibody produced by immunizing an animal with an immunogen, wherein the immunogen is an FIM as provided herein. In some embodiments, provided herein is an antibody generated by immunizing an animal with an immunogen, wherein the immunogen is an FIM as provided herein. In some embodiments, the animal is a human, a rabbit, a mouse, a hamster, a monkey, etc. In certain embodiments, the monkey is a cynomolgus monkey, a macaque monkey, or a rhesus macaque monkey. Immunizing the animal with an FIM immunogen can comprise, for example, administering at least one dose of a composition comprising the immunogen and optionally an adjuvant to the animal. In some embodiments, generating the antibody from an animal comprises isolating a B cell which expresses the antibody. Some embodiments further comprise fusing the B cell with a myeloma cell to create a hybridoma which expresses the antibody. In some embodiments, the antibody generated using the FIM can cross react with a human and a monkey, for example a cynomolgus monkey.

In certain embodiments, the method of generating an antibody further comprises determining one or more epitopes for the antibody. In some embodiments, the method comprises screening the antibody for binding to two or more epitopes, for example by contacting an epitope library with the antibody, and evaluating binding of the antibody to epitopes of the library. In certain embodiments, an antibody that binds to two or more epitopes is discarded. In some embodiments, the FIM mimics one epitope of the interface protein. In other embodiments, the FIM mimics two or more epitopes of the interface protein. In certain embodiments, screening an antibody for binding to two or more epitopes, wherein the FIM mimics two or more epitopes of the interface protein, comprises contacting an epitope library with the antibody, and evaluating binding of the antibody to epitopes of the library, and discarding one or more antibodies that binds to two or more epitopes, wherein the epitopes are not those mimicked by the FIM.

In some instances of the method of generating an antibody using an FIM, the method further comprises determining a biological effect for the antibody (e.g., an agonist antibody or an antagonist antibody). The biological effect may be, for example, inhibiting an activity of a target protein (for example, through competitive binding), increasing an activity of a target protein, inhibiting binding of a target protein to a binding partner, stabilizing binding of a target protein to a binding partner, increasing half-life of a target protein, or decreasing half-life of a target protein. In some embodiments, the target protein is the interface protein. Examples of a target protein that may be of interest include, for example, PD-1, PD-L1, CD25, IL2, MIF, or CXCR4. Thus, in some embodiments, the FIM is an immunogen that can be used to raise one or more antibodies that specifically bind to PD-1, PD-L1, CD25, IL2, MIF, or CXCR4. In some embodiments, the antibody is an agonist antibody.

In some embodiments, the FIM is a capture agent that can be used to isolate or bind to target molecules. In some instances, the FIMS captures specific proteins from a complex mixture, such as, for example, a biological sample, including blood, plasma, serum, urine, feces, cerebrospinal fluid, tissue and/or tissue extracts, and the like. In some embodiments, the FIM can be used to isolate proteins in phage library panning. In other embodiments, the FIM can be used to isolate proteins in yeast library panning. In some instances, the FIM is immobilized to a surface, for example, a microarray, a glass slide and/or bead complex. In yet other instances, the FIM captures antibody or other native or non-native binding partners of the target molecule on a functionalized surface. In some embodiments, the FIM is a therapeutic molecule. For example, in certain embodiments, the FIM is an antagonist for a therapeutic target. In other embodiments, the FIM is an agonist for a therapeutic target. In yet other embodiments, the FIM is a partial agonist or partial antagonist for a therapeutic target. Thus, in some embodiments, provided herein is a method of treating a disorder in a subject in need thereof, comprising administering to the subject a therapeutic amount of an FIM as described herein. In some embodiments, a therapeutic FIM molecule may be used in combination therapy. For example, the FIM, in some embodiments, may be co-administered with one or more other therapeutics. For such uses, an FIM may be administered at the same time as, or within a certain number of hours as, one or more other therapeutics.

In still further embodiments, the FIM can be used as a labeling reagent. For example, in some embodiments, the FIM is used to label a target in cells. In other embodiments, the FIM is used to label a target in one or more tissues. In some embodiments, the FIM is used as a labeling reagent (for example to label a target in cells or tissues) in vivo. In other embodiments, the FIM is used as labeling reagent in vitro. In certain embodiments, the FIM further comprises one or more additional functional moieties to assist in labeling, such as, for example, a fluorophore.

In some embodiments, the FIM is used as an enzyme substrate. In certain embodiments, the FIM is an enzyme substrate for modulating or screening novel enzyme activity. In some embodiments, the FIM is an enzyme substrate in certain microenvironments. For example, in some embodiments the FIM is an enzyme substrate in low-pH environments. In certain embodiments, the FIM is a protease substrate in a low-pH environment.

In some embodiments, the FIM is used as a post-translational modification surrogate. For example, in some embodiments, the FIM is a phosphorylation surrogate, for example for purposes of screening.

In other embodiments, the FIM is an enhancer of one or more pharmacokinetic properties. For example, in some embodiments, the FIM serves as an adjuvant, and is co-administered with one or more pharmaceutical compounds, such as a drug or a vaccine.

In some embodiments, the FIM exhibits Fc receptor (FcR) binding. In certain embodiments, the FIM exhibits binding to selective FcRs. Thus, in some embodiments, the FIM can be used for selective FcR engagement. For example, in some embodiments, the FIM is administered to subject in need thereof to stimulate an FcR. Thus, in some embodiments the FIM is used in one or more FcR driven activities.

In some embodiments, the FIM can be internalized by the cells of a subject, which may be useful, for example, in drug delivery applications. For example, in some embodiments, a drug is conjugated to the FIM either directly or through a linking moiety (such as a linker), and the conjugate administered to as subject in need thereof. In certain embodiments, at least a portion of the administered conjugate is internalized by one or more cells of the subject. In some embodiments, a greater proportion of the drug is internalized by the subject as a conjugate, than if the drug were administered alone.

In still further embodiments, the FIM is a carrier that can cross the blood-brain barrier. For example, in some embodiments, the FIM is conjugated to another molecule, such as a therapeutic or imaging molecule, and can serve as a carrier to transport the other molecule across the blood-brain barrier.

In some embodiments, the FIM can be used in neo-antigen screens for chimeric antigen receptor-T cell (CAR-T cell) applications. In some embodiments, the FIM is used to enhance CAR-T cell therapy.

In still other embodiments, the FIM can be used as a diagnostic reagent for capturing one or more biomarkers from a biofluid, such as blood or plasma.

In some embodiments, the FIM is covalently attached to an antibody. In certain embodiments, two or more FIMs are covalently attached to an antibody. In yet other instances, at least one FIM is attached to an Fc region of an antibody. In yet other instances, the Fc region of the antibody is of human origin.

II. Method of Making FIM Candidates and Selecting an FIM

In other aspects, provided herein is a method of selecting an FIM. In some embodiments, the structure of the interface protein is known before selecting the FIM. In other embodiments, the structure of the interface protein is not known before selecting the FIM. In still further embodiments, the interface protein structure is only partially known and/or poorly characterized before selecting the FIM. In certain embodiments, structural information from the interface protein is used in the method of selecting the FIM.

In some embodiments, the method of selecting an FIM comprises one or more molecular dynamics analysis steps. In some embodiments, an FIM or FIM candidate obtained using molecular dynamics analysis is used to generate one or more libraries of peptides, or one or more libraries of FIM candidates, and these libraries are screened for binding with a cognate binding partner of the target interface protein.

In other embodiments, the method of selecting an FIM comprises contacting a first library with a protein or fragment thereof, wherein the first library is a peptide library comprising a plurality of peptides. At least a portion of the peptides in the first library are selected based on binding with the protein or fragment thereof, and these selected peptides are used to generate a second library, wherein the second library is a combinatorial library comprising a plurality of FIM candidates. Each of these FIM candidates independently comprises at least two of the selected peptides connected by at least one linker. This combinatorial library is then contacted with a peptide or protein fragment thereof, and binding is evaluated. The FIM is selected from the combinatorial library based on binding with the protein or fragment thereof.

a. Molecular Dynamics

In some embodiments, provided herein are methods of selecting an FIM using molecular dynamics. In some embodiments, one or more sections of a target interface protein are identified as the target interface. For example, in some embodiments, a portion of the interface protein that is an epitope for one or more antibodies is identified as the target interface. In other embodiments, a portion of the interface protein that is not an epitope for one or more antibodies is identified as the target interface. In certain embodiments, one or more portions of the interface protein are identified as the target interface(s), and it is unknown if those portions bind to an antibody. In some embodiments, an FIM comprises interface residues retained from the target interface protein with intermediate, non-interface residues that fix the structure and dynamics relative to the cognate target structure and dynamics. In some embodiments, these non-interface residues are not from the target interface protein, or do not share one or more characteristics with the target interface protein, or share fewer characteristics and/or share characteristics less strongly with the target interface protein than the interface residues. These intermediate, non-interface residues may, in some embodiments, form part or all of an amino acid linker. In some embodiments of selecting an FIM using molecular dynamics, an initial design is produced and the molecular dynamics simulated to determine flexibility and overall stability of the design. If this initial design does not meet RMSD requirements, it undergoes iterative optimization of the intermediate linker residues using computational mutagenesis. The interface residues are fixed. The iterative optimization may be repeated until the FIM RMSD interface residue positions relative to the target interface and structural order metric meet certain requirements (for example, ≤6.0 Å and ≥0.25, respectively, wherein structural order is on a 0-1 normalized scale, where 1=perfect structural stability).

In some embodiments, the intermediate structural stability residue regions independently range from 1-50 amino acids in length. In certain embodiments, these intermediate structural stability residue regions are linkers, for example amino acid linkers. In some embodiments, the relatively small size of an FIM produced by certain embodiments of the methods provided herein (compared, for example, to approaches that graft an interface onto a large structurally stabilizing scaffold) may enable chemical synthesis of the molecule, in contrast to a larger molecule that may require an in vitro expression system. Furthermore, in some embodiments the methods provided herein enable the incorporation of non-natural amino acids into intermediate positions or the interface positions, which may allow for fine control of interface engineering with novel moieties and properties such as post-translational modifications, solubility, cell-permeability, enzyme reactivity, pH sensitivity, oxidation sensitivity, etc. In still further embodiments, FIMs may be selected with a higher likelihood of species cross-reactivity or disease-related mutation reactivity in selected antibodies when the FIM is used as an immunogen.

In some embodiments, the optimized molecule is the FIM. In other embodiments, the optimized molecule is an FIM candidate. In certain embodiments, the method includes using the FIM candidate or FIM to generate a peptide library, or an FIM candidate library, and then contacting the library with a cognate binding partner of the target interface protein. The peptide library may include, for example, peptides which are smaller than and share at least some sequence similarity with the FIM or FIM candidate, and in which certain residues are optionally replaced with other residues. AN FIM candidate library may include, for example, variations of the FIM candidate, for example with one or more additional linking moieties, or one or more linking moieties removed, or replacing one or more amino acid residues.

In some embodiments, the peptides of the library comprise between 2 to 15 amino acids, between 5 to 15 amino acids, between 10 to 15 amino acids, between 2 to 10 amino acids, or between 5 to 10 amino acids. In some embodiments, the total number of amino acids in each peptide of the library includes both the interface amino acids and structural amino acids, which may include, for example, linker amino acids. In still further embodiments, the FIM candidate or candidates are used to prepare an FIM candidate library. The library may be prepared by, for example, varying one or more amino acids or linking moieties in the candidates to make new library members. The FIM candidates in the FIM candidate library, in some embodiments, independently comprise between 5 to 40 amino acids, between 10 to 35 amino acids, between 15 to 35 amino acids, or between 20 to 30 amino acids. In some embodiments, the total number of amino acids in each FIM candidate of the FIM candidate library can, in some embodiments, include both the interface amino acids and structural amino acids, which may include, for example, linker amino acids. The FIM candidate library can, in some embodiments, provide additional information regarding the effect of certain linker moieties on binding interactions (including presence or location of such moieties), such as cross-links including disulfide bonds and lactams. The peptide or FIM candidate libraries, or both, may in some embodiments be used to identify common motifs (e.g., amino acid patterns or linking moieties, or combinations thereof) that may increase binding affinity or binding specificity for the cognate binding partner, or provide other desired characteristics. Evaluating the binding of the cognate binding partner with the members of the peptide or the FIM candidate libraries, or both, can provide additional structural and functional information, which may be used to further refine the FIM design or to select one of the FIM candidates. The peptide library and the FIM candidate library can, in some embodiments, independently comprise between 5,000 and 30,000 members, between 10,000 and 25,000 members, between 15,000 and 20,000 members, or about 17,000 members (e.g., distinct peptides or distinct FIM candidates). In some embodiments, the peptide library and the FIM candidate library (if present) independently have any of the characteristics described herein for libraries (e.g., size, composition, materials, etc.).

b. Peptide Library

In some embodiments of the methods of identifying an FIM provided herein, a peptide library is contacted by a protein or fragment thereof, and binding is evaluated to select a portion of the peptides to generate a combinatorial library. In some embodiments, the method further comprises generating the peptide library. In certain embodiments, a commercial peptide library is used.

In some embodiments, at least a portion of the peptides in the peptide library are designed. Peptides may be designed using structural information about a functional interface of the interface protein, or without structural information about a functional interface of the interface protein.

If structural information about the target interface (of the interface protein) is known, it can be used in some embodiments to design at least a portion of the peptides in the peptide library. Structural information may be obtained, for example, by x-ray crystallography, NMR, homology, or simulation, or any combinations thereof. In some embodiments, the peptide library is designed to include target-informative molecules by incorporating structure and sequence motifs relevant to the target interface protein. In some embodiments, the peptide library is designed to include primary structure characteristics of the target interface. In other embodiments, the peptide library is designed to include secondary structure characteristics of the target interface. For example, if it is known that the target interface has a relatively high prevalence of sheets or turns, the peptide library can be designed to include peptides with these secondary structural motifs by incorporating Trp-Zip into one or more peptides. In other embodiments, if it is known that the target interface has a relatively high prevalence of helical structures, the peptide library can be designed to include peptides with i,i+4 crosslinks. In still other embodiments, if it is known that the target interface has a relatively high prevalence of certain amino acid moieties, the peptide library is enriched with peptides that contain these moieties. In some embodiments, the amino acid moiety is selected from the group consisting of amine, carboxyl, alkyl, hydroxyl, aryl, and heteroaryl. In further embodiments, if it is known that the target interface has a relatively high prevalence of certain molecular properties, the peptide library is enriched with peptides that contain these properties. Examples of such properties can include, for example, hydrophobicity, charge, and nucleophilicity. For example, in some embodiments, the target interface has one or more functional groups, or one or more areas (such as formed by a plurality of functional groups), that are hydrophobic, hydrophilic, charged, protonated, unprotonated, negatively charged, hydrogen bond donating, hydrogen bond accepting, or nucleophilic. Thus, in some embodiments, one or more peptides of the peptide library can be designed to include hydrophobic, hydrophilic, charged, protonated, unprotonated, negatively charged, hydrogen bond donating, hydrogen bond accepting, or nucleophilic functional groups or areas to mimic the properties of the target interface. In some embodiments, a peptide has functional groups or areas that are hydrophobic, hydrophilic, charged, protonated, unprotonated, negatively charged, hydrogen bond donating, hydrogen bond accepting, or nucleophilic.

The peptide library may also in some embodiments be designed without using structural information about the target interface, for example if the structure of the interface is unknown or poorly characterized. In such embodiments, at least a portion of the first library may be designed using biases identified in the interactome of the interface protein, or biases identified in the interactome of the binding partner of the interface protein. Incorporating these biases in the design of the peptide library may, for example, increase the probability of success in identifying peptides that drive the protein interface interactions. Examples of target interactome bias may include but are not limited to: 1) Library enrichment with amino acid distribution known to exist at protein interfaces, or, in some embodiments, particular protein interface families; 2) Library enrichment with secondary structure distribution known to exist at protein interfaces, or, in some embodiments, particular protein interface families; 3) Library enrichment with chemical properties, (such as charge, hydrophobicity, protonation, hydrogen bonding, etc.), known to exist at protein interfaces or, in some embodiments, particular protein interface families.

In some embodiments, the primary library comprises at least about 50 peptides, at least about 100 peptides, at least about 1,000 peptides, at least about 5,000 peptides, at least about 10,000 peptides, at least about 20,000 peptides, at least about 30,000 peptides, at least about 40,000 peptides, at least about 50,000 peptides, at least about 75,000 peptides, at least about 100,000 peptides, at least about 125,000 peptides, at least about 150,000 peptides, at least about 175,000 peptides, at least about 200,000 peptides, at least about 250,000 peptides, at least about 500,000 peptides, at least about 750,000 peptides, at least about 1,000,000 peptides, at least about 2,000,000 peptides, or at least about 3,000,000 peptides. In certain embodiments, the primary library has a diversity of greater than about 10,000 distinct peptides.

In some embodiments, the methods of selecting an FIM provided herein comprise screening the peptide library and identifying one or more peptides that exhibit at least one characteristic of an interface protein, as described herein. In some embodiments, this screening is performed by contacting the peptide library with a protein or fragment thereof. In some embodiments, this protein is a cognate binding partner of the interface protein. In some embodiments, a fragment of a cognate binding partner protein is used. In certain embodiments, at least a portion of the peptides are selected based on their binding of the protein or fragment thereof. These selected peptides can then be used to generate a combinatorial library of FIM candidates, wherein each candidate independently comprises at least two peptides connected by at least one linker.

c. Combinatorial Library

In some embodiments of the methods of identifying an FIM provided herein, a combinatorial library comprising a plurality of FIM candidates is contacted by a protein or fragment thereof, and binding is evaluated to select the FIM. In some embodiments, FIM candidates each independently comprise at least two peptides selected from the peptide library, independently connected by at least one linker. In some embodiments, the combinatorial library comprises a plurality of FIM candidates the each independently comprise any of the peptides and linkers described herein.

In some embodiments, in addition to selecting peptides to form FIM candidates, one or more additional design considerations is used in generating at least portion of the combinatorial library members. Such design considerations may include, for example, producing a library that incorporates motifs of interest identified in the peptide screen. For example, peptides identified from the peptide library based on binding with the protein or fragment thereof may, in some embodiments, be further clustered into distinct groups using sequence or structural information, or a combinations thereof. These identified peptides may, in some embodiments, be analyzed to identify shared structural or functional motifs. Such shared motifs may comprise one or more of sequence, structure, and chemical characteristics that lead to protein interactions at the target interface, in certain embodiments. Thus, enriching the combinatorial library with selected peptides that have shared motifs may, in some embodiments, increase the number of highly active FIM candidates that mimic the target interface interactions.

In addition to peptide design considerations, in some embodiments one or more linkers is selected for use in the combinatorial library based on a desired functionality or structural aspect. Various functional and structural aspects of different linkers in FIM and FIM candidates have been described herein, and each of these aspects can be considered in designing the combinatorial library before screening. In some embodiments, a linker is selected for use in the combinatorial library because it comprises one or more functional or structural factors that may lead to effective binding with the protein or fragment thereof during library screening. In other embodiments, a linker is selected for use in the combinatorial library because it comprises one or more functional or structural factors that is desirable for the application of the FIM being developed. For example, in some embodiments an FIM is being selected for use in a particular application that requires a capture handle, or a label, certain solubility, proteolytic activity, or another characteristic. In such embodiments, one or more linkers for use in the combinatorial library are selected that comprise that desired characteristic.

Each peptide selected for use in the combinatorial library is an individual combinatorial element that can be used to create a library of distinct FIM candidates. If multiple linkers are selected, these are also additional combinatorial elements that further expand the diversity of the FIM candidate library, in certain embodiments. In some embodiments, the combinatorial library comprises between two to twelve combinatorial elements (as peptides and linkers), which are used to create FIM candidates. In some embodiments, the combinatorial library has a diversity of greater than 10 distinct FIM candidates, or greater than 50 distinct FIM candidates, or greater than 100 distinct FIM candidates. In certain embodiments, to create a combinatorial library with secondary structure, peptides with at least 6 amino acids are selected. In certain embodiments, to create a combinatorial library with secondary structure, peptides with an average length of 9 amino acids are selected.

d. Additional Library Components

The libraries described herein, including the peptide libraries and combinatorial libraries used in the methods provided herein, may independently take a variety of different forms. For example, in some embodiments the libraries are independently supported on a solid phase, or free in solution.

In certain embodiments, a library (such as the peptide library, or combinatorial library, or both) that is solid supported may be attached to any suitable solid surface. For example, in some embodiments, the surface is flat, concave, or convex, or comprises a mixture of shapes. In some embodiments, the library is supported by a well plate, such as a 96-well plate or a 384-well plate. For example, the library may be supported by multiple wells of a well plate, wherein at least a portion of the wells independently comprise one or more members of the library attached to the well surface. In other embodiments, the solid surface of a library is flat. For example, in some embodiments the library is supported by a glass slide (or a plurality of glass slides), or a silicon wafer (or a plurality of silicon wafers). In still further embodiments, the library is supported by a curved surface. For example, in some embodiments the library is supported on the surface of a bead, or on the surface of a plurality of beads. In still further embodiments, the library is supported by a mixture of surfaces, for example by one or more beads and a glass slide.

For a solid-supported library, the members of the library may be attached to the solid surface by a variety of ways. For example, the peptides or FIM candidates can, in some embodiments, be physically tethered to a solid surface by a connector molecule. For example, for members of a peptide library, the N- or the C-terminus of a peptide can be attached by a connector molecule to a solid surface. For members of a combinatorial library, the N- or C-terminus of a peptide component, or a functional group of a linker, can be attached by a connector molecule to a solid surface. A connector molecule can be, for example, a functional plurality or molecule present on the solid surface, such as an imide functional group, an amine functional group, a hydroxyl functional group, a carboxyl functional group, an aldehyde functional group, and/or a sulfhydryl functional group. In some embodiments, a connector molecule is a polymer. For example, in some embodiments the connector molecule is polyethylene glycol. In some embodiments a connector molecule is maleimide. In some embodiments, a connector molecule is glycine-serine-cysteine (GSC) or glycine-glycine-cysteine (GGC). In yet other embodiments, the connector molecule is hydroxymethyl benzoic acid, 4-hydroxy-2-methoxy benzaldehyde, or 4-sulfamoyl benzoic acid. A plurality of different connector molecules can also, in some embodiments, be used to connect library members to a solid surface, or mixture of solid surfaces.

For a solid-supported library, the solid surface may comprise any suitable material, or a mixture of materials. For example, in some embodiment the solid surface comprises glass, silicon, germanium, gallium arsenide, gallium phosphide, silicon dioxide, sodium oxide, silicon nitride, nitrocellulose, nylon, polytetraflouroethylene, polyvinylidendiflouride, polystyrene, polycarbonate, or one or more methacrylates, or any combinations thereof. In some embodiments, the glass is functionalized glass. The surface of a library can, in some embodiments, comprise semi-conductor wafers, for example derivatized silicon wafers, such as silicon wafers with aminosilane groups. In some embodiments, at least a portion of the surface is coated with a solid coating. For example, in some embodiments, at least a portion of the surface is coated with a coating to improve adhesion capacity of one or more library members, or reduce background adhesion of undesired components. In some embodiments, at least a portion of the surface is coated with an aminosilane-coating. In certain embodiments, the coated surface is a silicon wafer or a glass slide.

In other embodiments, the library is not solid-supported. For example, in some embodiments the library is free in solution. For a solution-phase library, the library may be present in a single aliquot of solution, or a plurality of aliquots. For example, in some embodiments the library is free in solution and distributed amongst a plurality of wells in one or more well plates. In certain embodiments, the wells containing the library each contain a separate member of the library, or independently comprise one or more members of the library, or at least some of the same library members are present in different wells. In certain embodiments, for a solution-phase library, the solution is aqueous. In other embodiments, the solution is non-aqueous. In still further embodiments, a plurality of different solutions are used, for example in a library distributed across multiple containers (such as wells). In other embodiments, in a library distributed across multiple containers, the same solution is used.

e. Binding Evaluation

In some embodiments, the method of selecting an FIM provided herein comprises evaluating the binding of an FIM candidate to a protein or fragment thereof. For example, in some embodiments, an FIM candidate library is screened for binding to a protein or fragment thereof, wherein each FIM candidate of the library comprises at least one peptide that exhibits at least one characteristic of an interface protein, and at least one linking moiety. In some embodiments, at least one FIM candidate of the library comprises one peptide that exhibits at least one characteristic of an interface protein, and one linking moiety, wherein the linking moiety is a cross-link. In some embodiments, the FIM candidate that is being evaluated was designed using iterative computational methods as described herein.

In some embodiments, the method of selecting an FIM provided herein comprises selecting peptides from a peptide library based on binding with a protein or fragment thereof, and selecting an FIM from a combinatorial library based on binding with a protein or fragment thereof.

In some embodiments, the protein or fragment thereof used to contact the peptide library is the same as the protein or fragment thereof used to contact the combinatorial library. In certain embodiments, a different protein or fragment thereof is used to contact the two libraries. In some embodiments, a protein or fragment thereof is used to contact the peptide library, and a different fragment of the same protein is used to contact the combinatorial library. In still further embodiments, a protein or fragment thereof is used to contact the combinatorial library, and a different fragment of the same protein is used to contact the peptide library.

Binding of the protein or fragment thereof with one or more peptides or FIM candidates (such as a member of a library) may be evaluated in various ways. In some embodiments, binding with one or more of the peptides of the peptide library is evaluated using the same method as binding of one or more FIM candidates of the combinatorial library. In other embodiments, binding of one or more FIM candidates of the combinatorial library is evaluated using a different method than binding of one or more of the peptides of the peptide library.

In some embodiments, binding of the protein or fragment thereof with a peptide or FIM candidate (such as a member of a library) is directly evaluated, for example by directly detecting a label on the protein or fragment thereof. Such labels may include, for example, fluorescent labels, such as a fluorophore or a fluorescent protein.

In other embodiments, binding of the protein or fragment thereof with a peptide or FIM candidate (such as a member of a library) is indirectly evaluated, for example using a sandwich assay. In a sandwich assay, a peptide or FIM candidate (such as a member of a library) binds to a binding partner (such as a protein or fragment thereof), and then a secondary labeled reagent is added to label the bound binding partner. This secondary labeled reagent is then detected. Examples of sandwich assay components include His-tagged-binding partner detected with an anti-His-tag antibody or His-tag-specific fluorescent probe; a biotin-labeled binding partner detected with labeled streptavidin or labeled avidin; or an unlabeled binding partner detected with an anti-binding-partner antibody.

In some embodiments, peptides or FIM candidates of interest are identified (such as from a peptide or combinatorial library, or an FIM candidate library) based on the binding signal, or dose-response, using any number of available detection methods. These detection methods may include, for example, imaging, fluorescence-activated cell sorting (FACS), mass spectrometry, or biosensors. In some embodiments, a hit threshold is defined (for example the median signal), and any with signal above that signal is flagged as a putative hit motif.

For the development of the combinatorial library, peptides identified from the peptide library based on binding with the protein or fragment thereof may, in some embodiments, be further clustered into distinct groups using sequence or structural information, or a combinations thereof. This grouping may be done, for example, using generally available sequence alignment, chemical descriptors, structural prediction, and entropy prediction informatics tools (e.g. MUSCLE, CLUSTALW, PSIPRED, AMBER, Hydropathy Calculator, and Isoelectric Point Calculator) and clustering algorithms (e.g., K-Means, Gibbs, and Hierarchical). Clusters of motifs (e.g., structural or functional motifs) present in peptide hits can be identified from this analysis. Individual peptide motif hits can also be identified. Using these motif clusters and individual motifs, in some embodiments, design rules can be formulated that define one or more of sequence, structure, and chemical characteristics of the motifs that appear to drive the protein interactions at the target interface. In some embodiments, the structure of the target interface is not necessary for identification of these interface motif design rules. Rather, the design rules can, in some embodiments, be derived from analysis of peptides identified from screening the peptide library.

In some embodiments, the binding assay has a sensitivity dynamic range of about 10⁵. Thus, in some embodiments, a peptide or FIM candidate identified as of interest based on the binding assay is one that has a binding event with the protein or fragment thereof that is within a 10⁵ signal bracket of the native protein-protein interface signal. The type of signal may be different depending on what type of assay is being used, or how it is being evaluated. For example, in some embodiments, the signal is response units in a sensorgram, fluorescence signal in an image-based readout, or enzymatic readout in an enzyme-based assay. The signal for binding events may be measured relative to the cognate protein-protein interaction, and the peptide or FIM candidate of interest identified.

In addition to binding interactions with a protein or fragment thereof (such as the cognate binding partner of the interface protein), one or more other characteristics may, in some embodiments, be considered when identifying a peptide or FIM candidate as of interest. In some embodiments, a peptide or FIM candidate is identified as of interest at least in part based on biophysical metrics. Biophysical metrics may include, for example, predicted structural stability based on molecular dynamics calculations, isoelectric point, predicted solubility, or predicted secondary structure content. In some embodiments, a peptide or FIM candidate is identified as of interest based at least in part on “learned” metrics from pre-trained machine learning algorithms. In some embodiments, a combination of metrics is used to rank peptides or FIM candidates. For example, in some embodiments, a combination of binding interactions, biophysical metrics, and/or learned metrics are used to rank peptides or FIM candidates. Thus, for example, in some embodiments, the method of selecting an FIM comprises contacting a peptide library with a protein or fragment thereof, selecting at least a portion of peptides based on binding with the protein or fragment thereof, and further evaluating one or more additional factors of the selected peptides to identify which peptides to use in generating the combinatorial library. In some embodiments, the method of selecting an FIM comprises contacting a library comprising FIM candidates (which may include a combinatorial library) with a protein or fragment thereof, and selecting an FIM from the library based on binding with the protein or fragment thereof and one or more additional factors. In certain embodiments of these methods, the one or more additional factors may include, for example, biophysical metrics such as predicted structural stability based on molecular dynamics calculations, isoelectric point, predicted solubility, or predicted secondary structure content; or learned metrics from pre-trained machine learning algorithms; or a combination thereof.

It should be understood that, in some embodiments, the peptide library or the combinatorial library may be screened for binding with the protein or fragment thereof in one or more steps, or portions of the library may be screened at a different time than the rest of the library, or the library may be screened multiple times. In some embodiments, an FIM candidate library may be screened for binding with the protein or fragment thereof in one or more steps, or portions of the library may be screened at a different time than the rest of the library, or the library may be screened multiple times. In some embodiments, one or more additional libraries is screened in a method of selecting an FIM. In some embodiments, one or more additional peptide libraries is screened, or one or more additional combinatorial libraries is screened, or a combination of additional libraries is screened to select an FIM. For example, in certain embodiments, a first peptide library is screened for binding with the protein or fragment thereof, and at least a portion of the peptides are selected and combined with additional peptides to form a second peptide library, and this section peptide library is screened for binding with the protein or fragment thereof to select peptides for forming a combinatorial library of FIM candidates. In other embodiments, a first peptide library is screened and at least a portion of the peptides are selected to create a first combinatorial library; a second peptide library is screened and at least a portion of the peptides are selected to create a second combinatorial library; the first and second combinatorial libraries are screened separately and at least a portion of the FIM candidates selected; and those selected FIM candidates are combined to form a third combinatorial library which is screened to identify the FIM.

f. Additional Steps

In certain embodiments, the methods provided herein comprise one or more additional steps.

In some embodiments the method further comprises one or more additional screening steps. For example, as discussed herein, in some embodiments one or more linking moieties (such as linkers) is included in the combinatorial library in order to impart upon the FIM candidates a desired function, such as a capture handle, or a label, or certain solubility, or proteolytic activity, or another characteristic. Thus, in some embodiments the methods provided herein further comprise a linker activity screening step. In some embodiments, the FIM candidate library (which may be a combinatorial library) is screened for linker activity. In certain embodiments, a portion of the FIM candidate library (which may be a combinatorial library) is screened for linker activity. In some embodiments, the FIM candidate library (which may be a combinatorial library) is contacted by a protein or fragment thereof, a portion of the FIM candidate library is selected based on binding with the protein or fragment thereof, and this portion is screened for linker activity. In some embodiments, the FIM is selected from the FIM candidate library (which may be a combinatorial library) based both on binding with the protein or fragment thereof and linker activity.

In other embodiments, the FIM candidate library (which may be a combinatorial library) is contacted by a protein or fragment thereof, a portion of the FIM candidate library is selected based on binding with the protein or fragment thereof, and the FIM candidates in this selected portion undergo additional screening for one or more target interface behaviors. For example, in some embodiments, the selected portion of the FIM candidate library undergoes additional evaluation for one or more of affinity, phenotype, or specificity features that mimic the target interface protein, or the target interface. In one example, the FIM candidate library comprises 100 or fewer FIM candidates; the FIM candidate library is contacted by a protein or fragment thereof, and a portion of the library is selected based on binding with the protein or fragment thereof, wherein the portion comprises 20 or fewer FIM candidates; the portion is evaluated for one or more features that mimics the target interface protein; and an FIM is selected from the FIM candidate library based on both binding with the protein or fragment thereof and the evaluation of one or more features that mimics the target interface protein. In some embodiments, the one or more features is affinity, phenotype, specificity, or a combination thereof. In certain embodiments, the FIM candidate library is a combinatorial library.

In some embodiments, an FIM is selected based, at least in part, on structural flexibility at physiological pH compared to structural flexibility at a different pH (For example, in selecting an FIM that binds with a target protein associated with cancer (e.g., a cancer epitope) it may be useful to select an FIM that is more rigid at lower pH, or in which one or more amino acids have a particular orientation at lower pH, or has one or more other structural characteristics at lower pH, compared to the same FIM at physiological pH. Selecting an FIM based on these criteria may, in some embodiments, result in higher specificity or affinity for said target protein, or better binding with said target protein. In many cancerous tumors, the growth rate of cancerous cells can outpace the oxygen supply available in portions of the tumor, resulting in a hypoxic microenvironment within the tumor. The level of oxygen in tissues can affect the pH of the tissue environment, and hypoxic levels can lead to decreased pH (including, for example, by the buildup of acidic metabolites from anaerobic glycolysis). Thus, in some embodiments, selecting FIMs that have greater binding at low pH (e.g., have desirable structural characteristics that lead to binding interactions), but have reduced binding at physiological pH (e.g., have decreased, fewer, or no desirable structural characteristics that lead to binding interactions), can, in some embodiments, result in FIMs that when used as immunogens to produce an antibody, the antibody has greater binding to the desired target in a tumor, compared to binding not in a tumor. Physiological pH is typically between about 7.35 and about 7.45, for example about 7.4. The pH of a tumor microenvironment may be, for example, less than about 7.45, less than about 7.45, between about 7.45 and about 6.0, between about 7.0 and about 6.0, between about 6.8 and about 6.2, between about 6.7 and about 6.3, about 6.4, about 6.5, about 6.6, about 6.7, about 6.8, about 6.9 or about 7.0

In some embodiments, selecting an FIM may include comparing the binding of the FIM to binding of an inverse FIM. An inverse FIM includes an FIM in which one or more of the interface-interacting amino acid residues is replaced with an amino acid that exhibits an inverse characteristic. For example, an amino acid with a large, sterically bulky, hydrophobic side chain may be replaced with an amino acid that has a smaller side chain, or hydrophilic side chain, or a side chain that is both smaller and hydrophilic. In some embodiments, an amino acid with a hydrogen bond-donating side chain may be replaced with an amino acid that has a hydrogen bond-accepting side chain, or with a an amino acid that has a side chain that does not hydrogen bond. Binding characteristics that may be compared using the FIM and the inverse FIM may include, in some embodiments, specificity and/or affinity. Comparing the binding characteristics of an FIM with the binding characteristics of an inverse FIM may, in some embodiments, help select FIMs in which the interface-interacting amino acids drive the binding interactions, rather than characteristics of a linking moiety such as a linker. FIMs in which binding is driven by a linking moiety such as a linker, and not by interface residues, may be less desirable in some embodiments as they may exhibit off-target binding or other undesirable binding characteristics.

In further embodiments, the method further comprises modifying the selected FIM.

The description herein sets forth numerous exemplary configurations, methods, parameters, and the like. It should be recognized, however, that such description is not intended as a limitation on the scope of the present disclosure, but is instead provided as a description of exemplary embodiments.

What is claimed is:

1. A functional interface mimic, comprising:
at least two peptides connected by at least one linker, wherein each peptide independently exhibits at least one characteristic of an interface protein; and
wherein the functional interface mimic exhibits at least one characteristic of the interface protein.

2. The functional interface mimic of claim 1, comprising 2 to 12 peptides.

3. The functional interface mimic of claim 1 or 2, comprising 4 peptides.

4. The functional interface mimic of any one of claims 1 to 3, wherein each peptide independently comprises 2 to 40 amino acids.

5. The functional interface mimic of any one of claims 1 to 4, wherein each peptide independently comprises 9 to 15 amino acids.

6. The functional interface mimic of any one of claims 1 to 5, wherein the interface protein has a cognate binding partner; wherein at least one of the at least one characteristics of the interface protein that the functional interface mimic exhibits is binding with the cognate binding partner;
and wherein the binding is within at least one order of magnitude of the interface protein-cognate binding partner binding.

7. The functional interface mimic of any one of claims 1 to 6, wherein the functional interface mimic is an immunogen, an antagonist, an agonist, or a capture agent.

8. A method of generating an antibody specific for a target, comprising injecting into an animal a functional interface mimic, wherein the functional interface mimic is an immunogen according to claim 7, and wherein the immunogen mimics the target.

9. A population of functional interface mimic candidates, wherein a plurality of the candidates each independently comprise at least two peptides connected by at least one linker; wherein each peptide independently exhibits at least one characteristic of an interface protein; and wherein the functional interface mimic exhibits at least one characteristic of the interface protein.

10. A method of selecting a functional interface mimic, comprising:
(a) contacting a peptide library with a protein or fragment thereof, wherein the peptide library comprises a plurality of peptides, and wherein the protein or fragment thereof binds with at least two peptides;
(b) selecting at least a portion of peptides based on binding with the protein or fragment thereof that contacted the peptide library;
(c) generating a combinatorial library comprising a plurality of functional interface mimic candidates, wherein each candidate independently comprises at least two selected peptides connected by at least one linker;
(d) contacting the combinatorial library with the protein or fragment thereof, wherein the protein or fragment thereof binds with at least one candidate; and
(e) selecting a functional interface mimic from the combinatorial library based on binding with the protein or fragment thereof that contacted the combinatorial library.

11. The method of claim 10, wherein the functional interface mimic exhibits at least one characteristic of an interface protein; wherein the interface protein has a cognate binding partner; wherein at least one of the at least one characteristics of the interface protein that the functional interface mimic exhibits is binding with the cognate binding partner; and wherein the binding is within at least one order of magnitude of the interface protein-cognate binding partner binding.

12. The method of claim 10 or 11, wherein the functional interface mimic is an immunogen, an antagonist, an agonist, or a reagent.

13. The method of any one of claims 10 to 12, wherein the peptide library comprises at least 10,000 peptides.

14. The method of any one of claims 10 to 13, wherein the functional interface mimic comprises 2 to 12 peptides.

15. The method of any one of claims 10 to 14, wherein each peptide independently comprises 2 to 40 amino acids.

16. The method of any one of claims 10 to 15, wherein each peptide independently comprises 9 to 15 amino acids.

17. The method of any one of claims 10 to 16, wherein the protein or fragment thereof that contacts the peptide library is the same protein or fragment thereof that contacts the combinatorial library.

18. A functional interface mimic, produced by:
(a) contacting a peptide library with a protein or fragment thereof, wherein the peptide library comprises a plurality of peptides, and wherein the protein or fragment thereof binds with at least two peptides;
(b) selecting at least a portion of peptides based on binding with the protein or fragment thereof that contacted the peptide library;
(c) generating a combinatorial library comprising a plurality of functional interface mimic candidates, wherein each candidate independently comprises at least two selected peptides connected by at least one linker;
(d) contacting the combinatorial library with a protein or fragment thereof, wherein the protein or fragment thereof binds with at least one candidate; and
(e) selecting the functional interface mimic from the combinatorial library based on binding with the protein or a fragment thereof that contacted the combinatorial library.

19. The functional interface mimic of claim 18, wherein the functional interface mimic is an immunogen, an antagonist, an agonist, or a reagent.

20. The functional interface mimic of claim 18 or 19, wherein the functional interface mimic exhibits at least one characteristic of an interface protein; wherein the interface protein has a cognate binding partner; wherein at least one of the at least one characteristics of the interface protein that the functional interface mimic exhibits is binding with the cognate binding partner;
and wherein the binding is within at least one order of magnitude of the interface protein-cognate binding partner binding.

21. The functional interface mimic of any one of claims 18 to 20, wherein the peptide library comprises at least 10,000 peptides.

22. The functional interface mimic of any one of claims 18 to 21, wherein the functional interface mimic comprises 2 to 12 peptides.

23. The functional interface mimic of any one of claims 18 to 22, wherein each peptide independently comprises 2 to 40 amino acids.

24. The functional interface mimic of any one of claims 18 to 23, wherein each peptide independently comprises 9 to 15 amino acids.

25. The functional interface mimic of any one of claims 18 to 24, wherein the protein or fragment thereof that contacts the peptide library is the same protein or fragment thereof that contacts the combinatorial library.

26. A functional interface mimic, comprising at least one peptide that exhibits at least one characteristic of an interface protein, and at least one linking moiety, wherein the functional interface mimic exhibits at least one characteristic of the interface protein.

27. The functional interface mimic of claim 26, wherein each linking moiety is independently a cross-link or linker.

28. The functional interface mimic of claim 27, wherein each cross-link is independently a disulfide bond or an amide bond.

29. The functional interface mimic of claim 27 or 28, wherein each linker is independently an amino acid linker, a polymer linker, or a small molecule that does not comprise an amino acid.

30. The functional interface mimic of any one of claims 26 to 29, comprising at least two linking moieties, wherein each linker is independently a cross-link or linker.

31. The functional interface mimic of any one of claims 26 to 30, comprising 2 to 12 peptides.

32. The functional interface mimic of any one of claims 26 to 31, comprising 4 peptides.

33. The functional interface mimic of any one of claims 26 to 32, wherein each peptide independently comprises 2 to 40 amino acids.

34. The functional interface mimic of any one of claims 26 to 33, wherein each peptide independently comprises 9 to 15 amino acids.

35. The functional interface mimic of any one of claims 26 to 34, wherein the interface protein has a cognate binding partner; wherein at least one of the at least one characteristics of the interface protein that the functional interface mimic exhibits is binding with the cognate binding partner; and wherein the binding is within at least one order of magnitude of the interface protein-cognate binding partner binding.

36. The functional interface mimic of any one of claims 26 to 35, wherein the functional interface mimic is an immunogen, an antagonist, an agonist, or a reagent.
